(19) United States
(12) Patent Application Publication
(10) Pub. No.: US 2023/0121932 A1
(43) Pub. Date: Apr. 20, 2023

(54) HEPATOPANCREATIC NECROSIS DISEASE SIGNATURES AND USES THEREOF

(71) Applicant: ARIZONA BOARD OF REGENTS ON BEHALF OF THE UNIVERSITY OF ARIZONA, Tucson, AZ (US)

(72) Inventors: Hung MAI, Tucson, AZ (US); Arun DHAR, Tucson, AZ (US); Luis Fernando ARANGUREN CARO, Tucson, AZ (US)

(21) Appl. No.: 17/794,549

(22) PCT Filed: Jan. 21, 2021
(86) PCT No.: PCT/US2021/014435

§ 371 Date: Jul. 21, 2022

Related U.S. Application Data
(60) Provisional application No. 62/964,047, filed on Jan. 21, 2020.

(51) Int. Cl. C12Q 1/6883; A61K 45/06

(52) U.S. Cl. CPC C12Q 1/6883; A61K 45/06; C12Q 2600/158

(57) ABSTRACT

Described herein are methods of detecting acute hepatopancreatic necrosis disease (AHPND) tolerant and AHPND susceptible organisms and uses thereof. Also described herein are signatures of AHPND tolerant and AHPND susceptible organisms.

### CROSS-REFERENCE TO RELATED APPLICATIONS

This application claims the benefit of and priority to co-pending U.S. Provisional Patent Application No. 62/964,047, filed on Jan. 21, 2020, entitled “HEPATOPANCREATIC NECROSIS DISEASE SIGNATURES AND USES THEREOF,” the contents of which is incorporated by reference herein in its entirety.

### SEQUENCE LISTING

This application contains a sequence listing filed in electronic form as an ASCII.txt file entitled UAZ-0105WP.txt, created on Jan. 21, 2021 and having a size of 7,000 bytes. The content of the sequence listing is incorporated herein in its entirety.

### TECHNICAL FIELD

The subject matter disclosed herein is generally directed to detecting hepatopancreatic necrosis disease in shellfish, such as shrimp, and more particularly detecting susceptible and resistant strains of animals.

### BACKGROUND

Fish and shellfish serve as a major source of revenue in many countries with large coastal boundaries in Asia and the Americas, which are largely responsible for the global aquaculture production. Additionally, seafood serves as a major source of protein in these countries. Among various aquaculture species, shrimp is a high value species that ranks second position in contributory value with a total of about $142 billion. Despite the importance of shrimp to the global economy and food source, shrimp farming has often been accomplished on the background of poor biosecurity measures and lack of attention to biosecurity warnings. As an inevitable result, the list of deadly diseases in shrimp aquaculture has been growing since the first report of a viral disease in shrimp, Baculovirus penaei (BP). As such there exists a need for the development of compositions, methods, and techniques for detection, prevention, treatment, and/or control of diseases effecting aquaculture, and more particularly, crustacean aquaculture.

Citation or identification of any document in this application is not an admission that such a document is available as prior art to the present invention.

### SUMMARY

Described in certain example embodiments herein are methods of detecting an acute hepatopancreatic necrosis disease (AHPND) and/or AHPND susceptible organisms and/or cells therefrom comprising detecting in one or more cells from an organism an AHPND signature, wherein the AHPND signature comprises
(a) one or more genes selected from the group consisting of: SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of: PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof
(e) one or more genes selected from the group consisting of: CLT1-like, SP, CHYB, CRST-P, PPEA2, and CHYA
(e) LGBP
(f) one or more genes selected from the group consisting of: CRST-P, SP, PPEA2, CHYA, and LGBP;
(g) one or more genes selected from the group consisting of: SP, CRST-P, PPAE2, CHYA; or
(h) combinations thereof;
   wherein detection of the AHPND signature indicates that the organism or cell(s) thereof is tolerant to AHPND or that the sample contains at least one cell that is tolerant to AHPND or detection of the AHPND signature indicates that the organism or cell(s) thereof is susceptible to AHPND or that the sample contains at least one cell that is susceptible to AHPND.

In certain example embodiments, the organism is a crustacean.

In certain example embodiments, the organism is a crab, lobster, crayfish, shrimp, prawn, or krill.

In certain example embodiments, the sample is obtained from a cell, an organ, a tissue, a bodily fluid, or a combination thereof.

In certain example embodiments, the sample is obtained from a hepatopancreas of the organism.

In certain example embodiments, the AHPND signature comprises
(a) decreased expression of SP as compared to a suitable control and increased expression of CHYA, LGBP, and PPEA2 as compared to a suitable control;
(b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal;
(c) increased expression of PPAE2 and CHYA as compared to a suitable control and decreased expression of CTL1-like, CRST-P, SP, and CHYB as compared to a suitable control;
(d) increased expression of CRST-P, PPAE2, and CHYA as compared to a control sample obtained from an AHPND tolerant animal;
(e) decreased expression of LGBP as compared to a suitable control
(f) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal
(g) increased expression of PPEA2, CHYA, and LGBP as compared to a suitable control and decreased expression of CRST-P and SP as compared to a suitable control
(h) increased expression of SP as compared to a control sample obtained from an AHPND susceptible animal and decreased expression of CRST-P, PPEA2, and CHYA as compared to a control sample obtained from an AHPND susceptible animal; or
(i) a combination thereof.

In certain example embodiments, the AHPND signature comprising (a), (b), (c), (d), or a combination thereof indicates that the organism or cell(s) thereof is susceptible to AHPND or that the sample contains at least one cell that is susceptible to AHPND.

In certain example embodiments, the AHPND signature comprising (e), (f), (g), (h), or a combination thereof indicates that the organism or cell(s) thereof is tolerant to AHPND or that the sample contains at least one cell that is tolerant to AHPND.

Described in certain example embodiments herein are methods of treating or preventing acute hepatopancreatic necrosis disease (AHPND) in an organism or population thereof comprising detecting an AHPND tolerant organism and/or AHPND susceptible organism as previously described in any one of paragraphs [0007]-[0014] and elsewhere herein and
(a) administering an effective amount of an agent effective to treat or prevent AHPND infection;
(b) administering an effective amount of an agent effective to increase the tolerance of the organism to AHPND and/or reduce the susceptibility of the organism to AHPND;
(c) perform techniques capable of reducing AHPND in the environment;
(d) perform techniques capable of decreasing the mortality of AHPND in the organism(s);
(e) perform techniques capable of reducing the spread of and/or introduction of AHPND into the organism or population thereof; or
(f) any combination thereof.

Described in certain example embodiments herein are methods of screening for an agent effective to modify the acute hepatopancreatic necrosis disease (AHPND) tolerance of an organism and/or modify the AHPND susceptibility of an organism, comprising contacting an AHPND tolerant cell or an AHPND susceptible cell having an initial cell signature and/or cell state or type with a test agent; and determining a change in the initial cell signature, a shift in initial cell state or type, or both, wherein a change in the initial cell signature, a shift in initial cell state or type or both identifies, the test agent as an agent effective to modify the AHPND tolerance or susceptibility of the cell, organism, or both, and wherein determining a change in the initial cell signature, a shift in initial cell state or type, or both comprises a method as previously described in any one of paragraphs [0007]-[0014] and elsewhere herein.

In certain example embodiments, the agent is effective to increase the AHPND tolerance, reduce AHPND of a cell, an organism, or both.

In certain example embodiments, the agent is effective to decrease the AHPND susceptibility of a cell, an organism, or both.

Described in certain example embodiments herein are cells, cell populations, or progeny thereof comprising an acute hepatopancreatic necrosis disease (AHPND) signature, wherein the AHPND signature comprises
(a) one or more genes selected from the group consisting of: SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of: PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof;
(e) one or more genes selected from the group consisting of: CLT1-like, SP, CHYB, CRST-P, PPEA2, and CHYA
(e) LGBP;
(f) one or more genes selected from the group consisting of: CRST-P, SP, PPEA2, CHYA, and LGBP;
(g) one or more genes selected from the group consisting of: SP, CRST-P, PPAE2, CHYA or;
(h) a combination thereof.

In certain example embodiments, the cell is a crustacean cell.

In certain example embodiments, the crustacean is a crab, lobster, crayfish, shrimp, prawn, or krill.

In certain example embodiments, the AHPND signature comprises
(a) decreased expression of SP as compared to a suitable control and increased expression of CHYA, LGBP, and PPEA2 as compared to a suitable control;
(b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal;
(c) increased expression of PPAE2 and CHYA as compared to a suitable control and decreased expression of CTL1-like, CRST-P, SP, and CHYB as compared to a suitable control;
(d) increased expression of CRST-P, PPAE2, and CHYA as compared to a control sample obtained from an AHPND tolerant animal;
(e) decreased expression of LGBP as compared to a suitable control;
(f) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal;
(g) increased expression of PPEA2, CHYA, and LGBP as compared to a suitable control and decreased expression of CRST-P and SP as compared to a suitable control;
(h) increased expression of SP as compared to a control sample obtained from an AHPND susceptible animal and decreased expression of CRST-P, PPEA2, and CHYA as compared to a control sample obtained from an AHPND susceptible animal; or
(i) a combination thereof.

In certain example embodiments, the AHPND signature comprising (a), (b), (c), (d), or a combination thereof indicates that the organism or cell(s) thereof is susceptible to AHPND or that the sample contains at least one cell that is susceptible to AHPND.

In certain example embodiments, the AHPND signature comprising (e), (f), (g), (h), or a combination thereof indicates that the organism or cell(s) thereof is tolerant to AHPND or that the sample contains at least one cell that is tolerant to AHPND.

Described in certain example embodiments herein are modified non-human organism comprising one or more modified genes or expression thereof, wherein the one or more genes are one or more genes of an AHPND signature and are
(a) one or more genes selected from the group consisting of: SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of: PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof;
(e) one or more genes selected from the group consisting of: CLT1-like, SP, CHYB, CRST-P, PPEA2, and CHYA;
(e) LGBP;
(f) one or more genes selected from the group consisting of: CRST-P, SP, PPEA2, CHYA, and LGBP;
(g) one or more genes selected from the group consisting of: SP, CRST-P, PPAE2, CHYA; or
(h) a combination thereof.

In certain example embodiments, the modified non-human organism is modified to comprise an AHPND tolerant signature.

In certain example embodiments, the AHPND tolerant signature comprises
(a) decreased expression of SP as compared to a suitable control and increased expression of CHYA, LGBP, and PPEA2 as compared to a suitable control;
(b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal;
(c) increased expression of PPAE2 and CHYA as compared to a suitable control and decreased expression of CTL1-like, CRST-P, SP, and CHYB as compared to a suitable control;
(d) increased expression of CRST-P, PPAE2, and CHYA as compared to a control sample obtained from an AHPND tolerant animal;
(e) decreased expression of LGBP as compared to a suitable control
(f) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal;
(g) increased expression of PPEA2, CHYA, and LGBP as compared to a suitable control and decreased expression of CRST-P and SP as compared to a suitable control;
(h) increased expression of SP as compared to a control sample obtained from an AHPND susceptible animal and decreased expression of CRST-P, PPEA2, and CHYA as compared to a control sample obtained from an AHPND susceptible animal; or
(i) a combination thereof.

In certain example embodiments, the modified non-human organism is a crustacean.

In certain example embodiments, the crustacean is a crab, lobster, crayfish, shrimp, prawn, or krill.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention can be utilized, and the accompanying drawings of which:

FIGS. 1A-1D—H&E (Mayer-Bennet hematoxylin and eosin-phloxine) histology of Penaeus vannamei from Bioassay 1. Penaeus vannamei from negative control tank from the P1 (Panel A) and P2 populations (Panel B). Acute phase infection of AHPND in shrimp from P1 population (Panel C). Terminal phase of AHPND infection in shrimp from P2 population (Panel D). Scale bars=10 μm.

FIG. 2—Comparative pirA- and pirB-toxin genes expression in challenged animals from Bioassay 2 in the P1 and P2 populations. The levels of mRNA expression is expressed as a normalized mean Ct value. The data is presented as ΔMean Ct±SD. Statistical significance between P1 and P2 populations for each of the candidate gene was determined using Student's t-test.

FIGS. 3A-3C—Gene expression profiles of metabolic and immune genes in shrimp Penaeus vannamei from Bioassay 2. (FIG. 3A) The mRNA expression profile in AHPND susceptible (P1) population following experimental challenge. (FIG. 3B) The mRNA expression profile in AHPND tolerant (P2) population following experimental challenge. Expression levels of each gene are shown relative to the expression in corresponding control treatment (FIG. 3C) Comparison of gene expression profile from AHPND susceptible (P1) vs. AHPND resistant/tolerant (P2) population. Expression levels of each gene are shown relative to the expression in P1 negative control treatment. The data is presented as log 2 of relative expression±SD. Statistical significance between control and challenged animals for each of the candidate gene was determined using Student's t-test. *=P<0.05. BGBP=β-glucan binding protein, CRST P=Crustin P, CTL=C-type lectin 1-like, ECSOD=Extracellular Copper/Zinc Superoxide dismutase, KPI=Kazal protease inhibitor, LGBP=Lipopolysaccharide and β-1,3-glucan-binding, PEN2=Penaidin 2, PPAE2=Prophenol oxidase activation system 2, SEP=Serpin8, SP=Serine protease, ChyA=Chymotrypsin A, ChyB=Chymotrypsin B.

FIGS. 4A-4C—Validation of gene expression profiles of metabolic and immune genes in Penaeus vannamei from Bioassay 3. The mRNA expression profile in P. vannamei AHPND susceptible (P1) population (FIG. 4A) and AHPND resistant/tolerant (P2) population (FIG. 4B) following experimental challenge. Expression level of each gene is shown relative to the expression in correspond control treatment (FIG. 4C) Comparison of gene expression profile from AHPND susceptible (P1) vs AHPND resistant/tolerant (P2) population. Expression levels of each gene are shown relative to the expression in P1 negative control treatment. The data are presented as log 2 of relative expression±SD. Statistical significance between control and challenged animals for each of the candidate gene was determined using Student's t-test. *=P<0.05. CRST P=Crustin P, CTL=C-type lectin 1-like, LGBP=Lipopolysaccharide and β-1,3-glucan-binding, PPAE2=Prophenol oxidase activation system 2, SP=Serine protease, CHYA=Chymotrypsin A, CHYB=Chymotrypsin B.

FIG. 5—Cumulative mortality percentage in two populations of Penaeus vannamei from Bioassay 1. P1=Population 1 (Susceptible); P2=Population 2 (Tolerant); SPF=Specific pathogen free; DPI=Days post-infection. The data is presented as Mortality Mean±SD.

FIGS. 6A-6B—Multiple alignment of amino acid sequences of PirA^{VP}/PirB^{VP} with Cry1Aa (AEI71570.1), Cry3Aa (AAU29411.1) and Cry3Bb (Q06117.1). (FIG. 6A) Multiple alignment of amino acid sequences of PirA^{VP} with Cry1Aa, Cry3Aa and Cty3Bb. (FIG. 6B) Multiple alignment of amino acid sequences of PirB^{VP} with Cry1Aa, Cry3Aa and Cry 3Bb. Dark gray bar underlining residues indicates identical residues. Black color indicates strong similarity. Light Grey color indicated weak similarity. The cleaved site for chymotrypsin was indicated by light gray bars under residues. The cleaved site for trypsin was indicated by medium gray bars under residues.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” can contain whole cells and/or live cells and/or cell debris. The biological sample can contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Biological samples, including but not limited to bodily fluids, can be obtained from an organism, for example by puncture, or other collecting or sampling procedures.

The terms “subject,” “individual,” and “patient” are used interchangeably herein to refer to a vertebrate, preferably a crustacean, and more preferably a shrimp, crayfish, and prawns. Mammals include, but are not limited to, murines, simians, humans, farm animals, sport animals, and pets. Crustaceans include, but are not limited to crabs, lobsters, crayfish, shrimps, prawns, krill, woodlice, and barnacles. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics can be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Acute hepatopancreatic necrosis disease (AHPND) is a lethal disease in marine shrimp that emerged initially in China in 2009 and then spread to other countries in South East Asia and the Americas. It is a disease that has caused over $1 billion in losses since its emergence. The etiologic agent was initially identified as Vibrio parahaemolyticus carrying plasmid-borne binary toxin genes, pirA and pirB (Han et al., 2015; Tran et al., 2013). Subsequently other species of Vibrio including V. harveyi, V. alginolyticus, and more recently Micrococcus luteus have been reported to cause AHPND (Duran-Avelar et al., 2018; Liu et al., 2016). The clinical signs of AHPND include atrophy and discoloration of hepatopancreas, soft shell, gut with discontinuous, and often 100% of mortality occurs in shrimp farms experiencing AHPND outbreak (Han et al., 2015; Lai et al., 2015; Soto-Rodriguez et al., 2014). Histopathology in AHPND animals reveal three different stages of disease development, namely initial, acute and terminal stages. In the initial phase, elongation of epithelial cells in hepatopancreas have been observed, whereas during the acute and terminal phase necrosis of tubular epithelial cells and inflammatory responses characterized by haemocytic infiltration are observed (Soto-Rodriguez et al., 2014). Hepatopancreatic malfunction and secondary bacterial infection are the contributing factors for mortality in infected shrimp (Han et al., 2015).

It is accepted that shrimp protect themselves from non-self objects by innate immunity that encompass humoral and cellular responses (Jiravanichpaisal et al., 2006). Recently, it has been reported that PirA- and PirB-like binary toxin encoded by V. parahaemolyticus can be neutralized by either hemocyanin or anti-lipopolysaccharide factor (Boonchuen et al., 2018; Maralit et al., 2018). Although shrimp immune responses to AHPND has been studied in relation to haemocyte and stomach epithelial cells (Boonchuen et al., 2018; Maralit et al., 2018; Soonthornchai et al., 2016), the immune response of shrimp to AHPND has not been elucidated in hepatopancreas that is accepted widely as a target organ of PirA- or PirB-like toxin (Han et al., 2015; Tran et al., 2013).

Currently, there is no effective therapy against AHPND. Antibiotics are not a viable treatment or prevention option as there is a prohibition on the use of antibiotics in farming shrimp. Thus, stocking ponds with Specific Pathogen Free (SPF) post-larvae, biosecurity and pond management remain the only effective strategies to mitigate AHPND epizootics. However, these practices significantly raise the cost of shrimp farming and do not address the ability to control infection if it does present in the population. As such, there exists an unmet need for compositions and techniques to control, treat, and/or prevent AHPND infection in shrimp.

With that said, embodiments disclosed herein can provide signatures, such as gene signatures, that can detect an AHPND tolerant organism and/or an AHPND susceptible organism. Also described herein are methods and assays that can be used to detect an AHPND tolerant organism and/or an AHPND susceptible organism. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Hepatopancreatic Necrosis Disease Signatures

Described herein are signatures, such as gene signatures, that can be used to detect an organism that is susceptible to or tolerant of AHPND. In general, such signatures are referred to herein as “AHPND signatures”, with specific signatures that are unique to AHPND susceptible organisms (AHPND susceptible signature) and specific signatures that are unique to AHPND tolerant (also referred to herein as “AHPND resistant” organisms). AHPND susceptible organisms are more susceptible to developing AHPND and/or infection by a causative agent of AHPND. AHPND tolerant organisms are those that are more resistant to developing AHPND and/or infection by a causative agent of AHPND and/or tolerate AHPND and/or infection by a causative agent of AHPND. The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status and/or phenotype of the entire cell (sub)population and/or organism. Furthermore, the signature can be indicative of cells within a population of cells in vivo. The signature can also be used to suggest, for instance, particular preventatives, therapies, combination disease control modalities or techniques, and/or to suggest ways to modulate one or more systems within a subject such as an immune or metabolic system. The AHPND signatures described herein can be determined by analysis of expression profiles of cell populations and/or of single-cells within a population of cells from isolated samples (e.g., hepatopancreatic samples), thus allowing determination of tolerant or susceptible organisms. The organism susceptibility to AHPND can be determined by specific signatures in cells (e.g., a susceptible or tolerant AHPND signature as previously discussed). The AHPND signatures described herein can also be characteristic of a specific cell state or type. The presence of the organism susceptibility to AHPND and any specific cell (sub)types or cell states can be determined by applying the signature genes to bulk sequencing data in a sample or by any other suitable method of gene and/or protein expression analysis.

Also described herein biomarkers (e.g., phenotype specific or cell type) that can be part of an AHPND signature and can be used in a method described herein in a variety of diagnostic, preventive, therapeutic, selective breeding and husbandry, and/or population screening and selection, particularly for AHPND susceptible and/or AHPND tolerant cells and organisms. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein. Specific biomarkers for AHPND tolerant and/or susceptible cells and/or organisms are described in greater detail elsewhere herein.

Biomarkers are useful in methods of determining AHPND susceptibility and/or tolerance in a subject by detecting a first level of expression, activity and/or function of one or more biomarkers and comparing the detected level or value to a control level or value, wherein a difference in the detected level and the control level indicates that the subject is susceptible to or tolerant of AHPND.

In some embodiments, distinct reference values can represent the prediction of a risk (e.g., an abnormally elevated risk) of being susceptible to or tolerant of AHPND or a causative infectious agent thereof as described elsewhere herein. In some embodiments, distinct reference values can represent predictions of differing degrees of tolerance of or susceptibility to AHPND.

Such comparison(s) can generally include any means to determine the presence or absence of at least one difference and optionally of the size of such difference between values being compared. A comparison can include a visual inspection (for example of an optical indicator), an arithmetical, or statistical comparison of measurements. Such statistical comparisons include, but are not limited to, applying a rule or set of rules.

Reference values can be established according to known procedures previously employed for other cell populations, biomarkers and gene or gene product signatures. For example, a reference value can be established in an individual or a population of organisms characterised by a particular level of tolerance of or susceptibility to AHPND disease or condition (i.e., for whom or what said diagnosis, prediction and/or prognosis of the AHPND holds true). Such population can include without limitation 2 or more, 10 or more, 100 or more, or even several hundred or more individual organisms or cells.

A “deviation” of a first value from a second value generally encompasses any direction (e.g., increase: first value > second value; or decrease: first value < second value) and any extent of alteration. For example, a deviation can encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation can encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

In some embodiments, a deviation can refer to a statistically significant observed alteration. For example, a deviation can refer to an observed alteration which falls outside of error margins of reference values in a given population (as expressed, for example, by standard deviation (SD) or standard error (SE), or by a predetermined multiple thereof, e.g., ±1×SD or ±2×SD or ±3×SD, or ±1×SE or ±2×SE or ±3×SE). Deviation can also refer to a value falling outside of a reference range defined by values in a given population (for example, outside of a range which includes ≥40%, ≥50%, ≥60%, ≥70%, ≥75% or ≥80% or ≥85% or ≥90% or ≥95% or even ≥100% of values in said population).

In some embodiments, a deviation is concluded if an observed alteration is beyond a given threshold or cut-off. Such threshold or cut-off can be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In some embodiments, the signature genes, biomarkers, and/or cells can be detected and/or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and can be used herein. detection can include primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25). Other suitable detection methods are described elsewhere herein.

Not being bound by a theory, the signatures of the present invention can be microenvironment specific, such as their expression in a particular spatio-temporal context, or in response to exposure to an etiological agent of AHPND. Not being bound by a theory, signatures as discussed herein are specific to a particular pathological context. Not being bound by a theory, a combination of cell subtypes having a particular signature can indicate an outcome. Not being bound by a theory, the signatures can be used to deconvolute the network of cells present in a particular pathological condition and/or susceptibility to and/or tolerance of AHPND. Not being bound by a theory the presence of specific cells and cell subtypes can be indicative of a particular response to a pathological agent(s) (e.g. an etiological agent(s) of AHPND), such as including increased or decreased susceptibility to the pathological agent(s). The signature can indicate the presence of one particular cell type. In one embodiment, the novel signatures are used to detect multiple cell states or hierarchies that occur in subpopulations of cancer cells that are linked to a particular pathological condition and/or susceptibility to and/or tolerance of AHPND or linked to a particular outcome or progression of the disease, or linked to a particular response to a prevention and/or treatment of the disease.

In some embodiments, the AHPND signature described herein is composed in whole or at least in part of one or more genes, proteins and/or epigenetic elements, such as for instance 1, 2, 3, 4, 5, 6, 7, 8, 9, 10, 11, 12 or more genes, proteins and/or epigenetic elements. In some embodiments, the AHPND signature described herein is composed in whole or at least in part of one or more genes, proteins and/or epigenetic elements, such as for instance 2, 3, 4, 5, 6, 7, 8, 9, 10, 11, 12 or more. In some embodiments, the AHPND signature described herein is composed in whole or at least in part of one or more genes, proteins and/or epigenetic elements, such as for instance 3, 4, 5, 6, 7, 8, 9, 10, 11, 12 or more. In some embodiments, the AHPND signature described herein is composed in whole or at least in part of one or more genes, proteins and/or epigenetic elements, such as for instance 4, 5, 6, 7, 8, 9, 10, 11, 12 or more. In some embodiments, the AHPND signature described herein is composed in whole or at least in part of one or more genes, proteins and/or epigenetic elements, such as for instance 5, 6, 7, 8, 9, 10, 11, 12 or more. In some embodiments, the AHPND signature described herein is composed in whole or at least in part of one or more genes, proteins and/or epigenetic elements, such as for instance 6, 7, 8, 9, 10, 11, 12 or more. In some embodiments, the AHPND signature described herein is composed in whole or at least in part of one or more genes, proteins and/or epigenetic elements, such as for instance 7, 8, 9, 10, 11, 12, or more. In some embodiments, the AHPND signature described herein is composed in whole or at least in part of one or more genes, proteins and/or epigenetic elements, such as for instance 8, 9, 10, 11, 12 or more. In some embodiments, the AHPND signature described herein is composed in whole or at least in part of one or more genes, proteins and/or epigenetic elements, such as for instance 9, 10, 11, 12 or more. In some embodiments, the AHPND signature described herein is composed in whole or at least in part of one or more genes, proteins and/or epigenetic elements, such as for instance 13, 14, 15, or more. It is to be understood that an AHPND signature described herein can for instance also include genes or proteins as well as epigenetic elements combined.

In certain embodiments, an AHPND signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, an AHPND signature contains one or more differentially expressed genes/proteins or differential epigenetic elements when comparing different cells or cell (sub)populations, including comparing different AHPND susceptible cells or AHPND susceptible cell (sub)populations, comparing different AHPND tolerant cells or AHPND tolerant cell (sub)populations, as well as comparing AHPND susceptible cells or AHPND susceptible cell (sub)populations with AHPND tolerant cells or AHPND tolerant cell (sub)populations. It is to be understood that “differentially expressed” genes/proteins include genes/proteins which are up- or downregulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression can be determined based on common statistical tests, as is known in the art.

As discussed herein, differentially expressed genes/proteins, or differential epigenetic elements can be differentially expressed on a single cell level or can be differentially expressed on a cell population level. In some embodiments, the differentially expressed genes, proteins, and/or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type which can be distinguished or are uniquely identifiable and set apart from other cells of this cell type. The cell subpopulation can be phenotypically characterized and, in some embodiments, is characterized by the signature as discussed herein. A cell (sub)population as referred to herein can constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

A gene signature can contain one or more genes or gene transcripts (simply transcripts) of interest. A transcript of interest can also be referred to interchangeably as a gene of interest or target sequence. Target sequence can refer to any polynucleotide, such as DNA or RNA polynucleotides. In some embodiments, a target sequence is derived from the nucleus or cytoplasm of a cell, and can include nucleic acids in or from mitochondrial, organelles, vesicles, liposomes or particles present within the cell and subjected to a single cell sequencing method, retaining identification of the source cell or subcellular organelle.

A gene of interest can include, for example, a mutation, deletion, insertion, translocation, single nucleotide polymorphism (SNP), splice variant or any combination thereof associated with a particular attribute in a gene of interest. A gene of interest is a gene involved in increasing the tolerance (or decreasing the susceptibility of) of an organism, cell thereof, or progeny thereof to AHPND and/or the infectious agent that causes AHPND.

Any gene, region or mutation of interest can be used to identify cells containing specific genes, regions or mutations, deletions, insertions, indels, or translocations of interest can be included in a library, such as a sequencing library. A gene of interest can be, for example, an AHPND signature gene, which refers to a gene that is differentially expressed between two different cells, organisms, or populations that have or is characteristic of different susceptibility and/or tolerance to an agent(s) that is/are capable of causing AHPND and/or developing AHPND when infected by an AHPND causative agent. In some embodiments, the AHPND signature gene is an immune system and/or metabolism gene. In some embodiments, the gene(s) of interest can be any one or more of those set forth in Table 1. In some embodiments, the gene(s) of interest can be SEP8 (KU853046.1), BGBP (AY249858.1), CRST-P (AY488497), CTL1-like (DQ858900.1), KPI (AY544986), LGBP (EU102286.1), EC-SOD (HM371157), SP (AY368151), PEN2 (Y14925), PPAE2 (AWF98992.1), CHYA (Y10664), CHYB (Y10665), and any combination thereof.

Thus, in some embodiments, an AHPND signature can include one or more AHPND signature gene(s) or gene products, which refers to gene(s) and gene product(s) that is/are differentially expressed between two different cells, organisms, or populations that have or is characteristic of different susceptibility or tolerance to an agent(s) that is/are capable of causing AHPND and/or developing AHPND once infected by an AHPND causative. As used herein, “gene product” refers to any product produced from (either directly, such as a transcript, or indirectly such as a protein) a gene. In some embodiments, the AHPND signature can include one or more immune system and/or metabolism gene(s) or gene program(s). In some embodiments, the AHPND signature can include one or more of those genes set forth in Table 1. In some embodiments, the AHPND signature can include/or be SEP8 (KU853046.1), BGBP (AY249858.1), CRST-P (AY488497), CTL1-like (DQ858900.1), KPI (AY544986), LGBP (EU102286.1), EC-SOD (HM371157), SP (AY368151), PEN2 (Y14925), PPAE2 (AWF98992.1), CHYA (Y10664), CHYB (Y10665), and any combination thereof.

In some embodiments, the AHPND signature is composed of
(a) one or more genes selected from the group consisting of: SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of: PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof;
(e) one or more genes selected from the group consisting of: CLT1-like, SP, CHYB, CRST-P, PPEA2, and CHYA;
(e) LGBP;
(f) one or more genes selected from the group consisting of: CRST-P, SP, PPEA2, CHYA, and LGBP;
(g) one or more genes selected from the group consisting of: SP, CRST-P, PPAE2, CHYA; or
(h) combinations thereof.

In certain example embodiments, the AHPND signature is composed of
(a) decreased expression of SP as compared to a suitable control and increased expression of CHYA, LGBP, and PPEA2 as compared to a suitable control;
(b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal;
(c) increased expression of PPAE2 and CHYA as compared to a suitable control and decreased expression of CTL1-like, CRST-P, SP, and CHYB as compared to a suitable control;
(d) increased expression of CRST-P, PPAE2, and CHYA as compared to a control sample obtained from an AHPND tolerant animal;
(e) decreased expression of LGBP as compared to a suitable control;
(f) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal;
(g) increased expression of PPEA2, CHYA, and LGBP as compared to a suitable control and decreased expression of CRST-P and SP as compared to a suitable control;
(h) increased expression of SP as compared to a control sample obtained from an AHPND susceptible animal and decreased expression of CRST-P, PPEA2, and CHYA as compared to a control sample obtained from an AHPND susceptible animal; or
(i) a combination thereof.

In certain example embodiments, the AHPND signature composed of (a), (b), (c), (d), or a combination thereof indicates that the organism or cell(s) thereof is susceptible to AHPND or that the sample contains at least one cell that is susceptible to AHPND.

In certain example embodiments, the AHPND signature composed of (e), (f), (g), (h), or a combination thereof indicates that the organism or cell(s) thereof is tolerant to AHPND or that the sample contains at least one cell that is tolerant to AHPND.

In some embodiments, the marker gene(s) of interest can include one or more mutations. In some embodiments, the mutations can cause or contribute to any observed differential expression and/or characteristic phenotype of a specific cell type, cell state, and/or organism (e.g., susceptibility to a AHPND pathological agent(s) (e.g., those agents that can result in and/or cause AHPND)). In some instances, the mutation is located anywhere in the gene. The gene of interest can include one or more single-nucleotide polymorphisms (SNP). The methods described elsewhere herein can be designed to distinguish SNPs within a population and thus can be used to distinguish susceptible strains that differ by a single SNP or detect certain disease and/or AHPND susceptibility specific SNPs, such as but not limited to, disease associated SNPs, such as without limitation AHPND associated SNPs.

Methods of Detecting Organisms Susceptible and/or Tolerant to AHPND

Generally, the methods described herein can be used to analyze and detect an AHPND signature that is characteristic of a cell, cell population, and/or organisms that are susceptible and/or tolerant to a pathological agent(s), such as the causative agent(s) of AHPND and/or the developing AHPND once infected by an AHPND causative agent. In some embodiments, the methods described herein can be used to stratify organisms into different populations based on their susceptibility (or tolerance) to a pathological agent(s), such as the causative agent(s) of AHPND and/or their susceptibility (or tolerance) to developing AHPND once infected with an AHPND causative agent. Described herein are methods and assays capable of detecting an AHPND signature in a cell, cell population, and/or organism. In some embodiments, the organism is a crustacean. In some embodiments, the crustacean is a shrimp, prawn, or crawfish.

In some embodiments, the method can include the step of detecting an AHPND signature, which is described in greater detail elsewhere herein, in a biological sample or a component thereof obtained from a subject or cell(s) thereof. In some embodiments, the subject is a crustacean. In some embodiments, the crustacean is a shrimp, prawn, or crawfish. In some embodiments, the sample is obtained from the hepatopancreas.

In some embodiments, an AHPND susceptible subject is detected when the subject has increased or upregulated expression, decreased or downregulated expression, or both of one or more genes of the measured gene signature as compared to a suitable control. In some embodiments, an AHPND tolerant subject is detected when the subject has increased or upregulated expression, decreased or downregulated expression, or both of one or more genes of the measured gene signature as compared to a suitable control. A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In some embodiments, the suitable control is a sample prepared from known AHPND susceptible strain of crustacean, such as shrimp, prawn or crawfish. In some embodiments, the suitable control is a sample prepared from known AHPND tolerant strain of crustacean, such as shrimp, prawn or crawfish. An exemplary susceptible strain of AHPND susceptible shrimp is P1 shrimp described herein. An exemplary strain of AHPND tolerant shrimp is P2 shrimp described herein. In some embodiments, the suitable control is a sample prepared from known AHPND tolerant strain of crustacean, such as shrimp, prawn or crawfish.

In some embodiments, an AHPND susceptible subject is characterized in that it has increased expression of LGPB, PPAE2, CHYA, CRST-P, CTL1-like, and any combination thereof as compared to a suitable control, where the suitable control is a sample prepared from an AHPND tolerant organism. In some embodiments, an AHPND tolerant subject is characterized in that it has decreased expression of LGPB, PPAE2, CHYA, CRST-P, CTL1-like, and any combination thereof as compared to a suitable control, where the suitable control is a sample prepared from an AHPND susceptible organism.

In some embodiments an AHPND susceptible subject is characterized in that it has decreased expression of CHYB, SP, and any combination thereof as compared to a suitable control, where the suitable control is a sample prepared from an AHPND tolerant organism. In some embodiments, a tolerant subject is characterized in that it has increased expression of CHYB, SP, and any combination thereof as compared to a suitable control, where the suitable control is a sample prepared from an AHPND susceptible organism.

In some embodiments, an AHPND susceptible subject is characterized in that it has a) increased expression of LGPB, PPAE2, CHYA, CRST-P, CTL1-like, and any combination thereof; b) decreased expression of CHYB, SP, and any combination thereof; or c) both as compared to a suitable control, where the suitable control is a sample prepared from an AHPND tolerant organism.

In some embodiments, an AHPND susceptible subject is characterized in that it has (a) decreased expression of SP as compared to a suitable control and increased expression of CHYA, LGBP, and PPEA2 as compared to a suitable control; (b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal; (c) increased expression of PPAE2 and CHYA as compared to a suitable control and decreased expression of CTL1-like, CRST-P, SP, and CHYB as compared to a suitable control; (d) increased expression of CRST-P, PPAE2, and CHYA as compared to a control sample obtained from an AHPND tolerant animal; or (e) a combination thereof.

In some embodiments, an AHPND tolerant subject is characterized in that it has (a) decreased expression of LGBP as compared to a suitable control; (b) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal; (c) increased expression of PPEA2, CHYA, and LGBP as compared to a suitable control and decreased expression of CRST-P and SP as compared to a suitable control; (d) increased expression of SP as compared to a control sample obtained from an AHPND susceptible animal and decreased expression of CRST-P, PPEA2, and CHYA as compared to a control sample obtained from an AHPND susceptible animal; or (e) a combination thereof.

Any suitable techniques of detecting expression of an AHPND susceptibility signature as described herein (e.g., gene, protein, epigenetic, etc. signature) can be used. Non-limiting examples of suitable techniques are described herein.

Described in certain example embodiments herein are methods of detecting an acute hepatopancreatic necrosis disease (AHPND) and/or AHPND susceptible organisms and/or cells therefrom including detecting in one or more cells from an organism an AHPND signature, wherein the AHPND signature is composed of
(a) one or more genes selected from the group consisting of: SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of: PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof;
(e) one or more genes selected from the group consisting of: CLT1-like, SP, CHYB, CRST-P, PPEA2, and CHYA;
(e) LGBP;
(f) one or more genes selected from the group consisting of: CRST-P, SP, PPEA2, CHYA, and LGBP;
(g) one or more genes selected from the group consisting of: SP, CRST-P, PPAE2, CHYA; or
(h) combinations thereof;

wherein detection of the AHPND signature indicates that the organism or cell(s) thereof is tolerant to AHPND or that the sample contains at least one cell that is tolerant to AHPND or detection of the AHPND signature indicates that the organism or cell(s) thereof is susceptible to AHPND or that the sample contains at least one cell that is susceptible to AHPND.

In certain example embodiments, the organism is a crustacean.

In certain example embodiments, the organism is a crab, lobster, crayfish, shrimp, prawn, or krill.

In certain example embodiments, the sample is obtained from a cell, an organ, a tissue, a bodily fluid, or a combination thereof.

In certain example embodiments, the sample is obtained from a hepatopancreas of the organism.

In certain example embodiments, the AHPND signature includes
(a) decreased expression of SP as compared to a suitable control and increased expression of CHYA, LGBP, and PPEA2 as compared to a suitable control;
(b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal;
(c) increased expression of PPAE2 and CHYA as compared to a suitable control and decreased expression of CTL1-like, CRST-P, SP, and CHYB as compared to a suitable control;
(d) increased expression of CRST-P, PPAE2, and CHYA as compared to a control sample obtained from an AHPND tolerant animal;
(e) decreased expression of LGBP as compared to a suitable control;
(f) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal;
(g) increased expression of PPEA2, CHYA, and LGBP as compared to a suitable control and decreased expression of CRST-P and SP as compared to a suitable control;
(h) increased expression of SP as compared to a control sample obtained from an AHPND susceptible animal and decreased expression of CRST-P, PPEA2, and CHYA as compared to a control sample obtained from an AHPND susceptible animal; or
(i) a combination thereof.

In certain example embodiments, the AHPND signature composed of (a), (b), (c), (d), or a combination thereof indicates that the organism or cell(s) thereof is susceptible to AHPND or that the sample contains at least one cell that is susceptible to AHPND.

In certain example embodiments, the AHPND signature composed of (e), (f), (g), (h), or a combination thereof indicates that the organism or cell(s) thereof is tolerant to AHPND or that the sample contains at least one cell that is tolerant to AHPND.

Suitable techniques include, but are not limited to, an RNA-seq method or technique, an immunoaffinity-based method or technique (e.g., immunohistochemistry, immunocytochemistry, immunoseparation assay, Western analysis, and the like), a polynucleotide sequencing method or technique (e.g., Maxium-Gilbert sequencing, chain-termination sequencing (e.g., Sanger sequencing), shotgun sequencing methods and techniques, bridge PCR, massively parallel signature sequencing, polony sequencing, pyrosequencing, Solexa sequencing, combinatorial probe anchor synthesis, SOLiD sequencing, Ion torrent semiconductor sequencing, nanoball sequencing, heliscope single molecule sequencing, single molecule real time sequencing, nanopore sequencing, microfluidic system-based sequencing, tunneling currents sequencing, sequencing by hybridization, sequencing with mass spectrometry, a RNA polymerase based-sequencing method, an in vitro virus high-throughput method, a bisulfite sequencing technique, or a combination thereof), a PCR based method or technique (e.g., PCR, RT-PCR, qPCR, RT-qPCR, etc.), a protein analysis technique (e.g., mass spectrometry, polypeptide sequencing, an immunoaffinity method or technique, and the like), an epigenome analysis technique, and combinations thereof. Other suitable methods and techniques will be appreciated by those of ordinary skill in the art. In some embodiments, the technique or method can measure the expression at the single-cell level. In some embodiments, the technique is a single-cell RNA-seq method or technique.

Biomarker detection can also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results can be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests can be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see e.g., ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they can absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

In some embodiments, the method can include or be based on a hybridization assay. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation can include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. The presence of hybridized complexes is then detected, either qualitatively or quantitatively. Specific hybridization technology which can be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as International Patent Application Publication WO 95/21265; WO 96/31622; WO 97/10365; WO 97/27317; and European Patent Application Publication EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, can be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-Interscience, NY (1987), which is incorporated in its entirety for all purposes. When the cDNA microarrays are used, typical hybridization conditions are hybridization in 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1SSC plus 0.2% SDS) (see e.g., Shena et al., Proc. Natl. Acad. Sci. USA, Vol. 93, p. 10614 (1996)). Useful hybridization conditions are also provided in, e.g., Tijessen, Hybridization with Nucleic Acid Probes”, Elsevier Science Publishers B.V. (1993) and Kricka, “Nonisotopic DNA Probe Techniques”, Academic Press, San Diego, Calif. (1992).

In certain embodiments, single cell RNA sequencing can be used (see e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the method can include plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the method can include high-throughput single-cell RNA-seq (see e.g., Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application publication WO2016168584A1; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the method can include performing single nucleus RNA sequencing to detect and/or measure an amount of a marker described elsewhere herein (see e.g., Swiech et al., 2014, Nature Biotechnology Vol. 33, pp. 102-106; Habib et al., 2016, Science, Vol. 353, Issue 6302, pp. 925-928; Habib et al., 2017, “Massively parallel single-nucleus RNA-seq with DroNc-seq” Nat Methods. 2017 October; 14(10):955-958; and International Patent Application Publication WO2017164936, which are herein incorporated by reference in their entirety).

In certain embodiments, the method can include performing involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

Other suitable assays, methods, and techniques for detecting and/or measuring an amount of a marker described elsewhere herein will be appreciated by one of ordinary skill in the art in view of the description herein.

In some embodiments, differences between AHPND tolerant and AHPND susceptible cells and/or organisms can be determined by using any of the methods described herein. In some embodiments, differences between AHPND tolerant signature and AHPND susceptible signature of cells and/or organisms can be determined by using any of the methods described herein.

In some embodiments, differences between AHPND tolerant and AHPND susceptible cells and/or organisms can determined by a method described herein, which can include comparing a gene expression distribution of cells from a test subject with a gene expression distribution of AHPND tolerant cells, AHPND susceptible cells, AHNPND diseased cells, non-diseased cells, reference, and/or other suitable control as determined by a gene expression, protein expression method and/or or another suitable method described herein.

In some embodiments, the method can include assessing the cell types, subtypes, and/or states present in the sample, which can include analyzing of expression matrices from expression data, performing dimensionality reduction, graph-based clustering and deriving list of cluster-specific genes in order to identify cell types and/or states present in the in vivo system and/or organism. These marker genes can then be used throughout to relate one cell state to another. For example, these marker genes can be used to relate an AHPND susceptible cell (sub)types and/or states to an AHPND tolerant or non-diseased cell (sub(types) and/or states (and vice versa). The same analysis can then be applied to the source material for the sample or a control. From both sets of the expression analysis an initial distribution of gene expression data is obtained. In certain embodiments, the distribution can be a count-based metric for the number of transcripts of each gene present in a cell. Further the clustering and gene expression matrix analysis allow for the identification of key genes in the non-diseased and/or AHPND tolerant cell-state and the AHPND susceptible cell state, such as differences in the expression of key transcription factors. In certain example embodiments, this can be done conducting differential expression analysis. Other analytic methods can be included or performed on their own. Detection and/or analysis is also described in the Working Examples herein.

In some embodiments, identification of an AHPND tolerant and/or AHPND susceptible cell or cell population can include detecting a shift, such as a statistically significant shift, in the cell-state as indicated by a modulation (e.g., an increased distance) in the gene expression space between a first cancer cell-state and a second cancer cell state and/or a normal or non-diseased cell. In certain embodiments, the distance is measured by a Euclidean distance, Pearson coefficient, Spearman coefficient, or combination thereof.

In certain embodiments, the gene expression space includes 5 or more genes, 10 or more genes, 20 or more genes, 30 or more genes, 40 or more genes, 50 or more genes, 100 or more genes, 500 or more genes, or 1000 or more genes. In some embodiments, the gene expression space includes 12 genes. In certain embodiments, the expression space defines one or more cell pathways. In some embodiments, the expression space is composed of one or more genes from Table 1. In some embodiments, the expression space is composed of SEP8, BGBP, CRST-P CTL1-like, KPI, LGBP, EC-SOD, SP, PEN2, PPAE2, CHYA, CHYB, and any combination thereof.

The statistically significant shift can be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%. The statistical shift can include the overall transcriptional identity or the transcriptional identity of one or more genes, gene expression cassettes, or gene expression signatures of the a first cancer cell state compared to a second cancer cell state and/or a normal or non-diseased state (i.e., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% of the genes, gene expression cassettes, or gene expression signatures are statistically shifted in a gene expression distribution). A shift of 0% means that there is no difference to the cell states.

A gene distribution can be the average or range of expression of particular genes, gene expression cassettes, or gene expression signatures in an AHPND tolerant cell-state, an AHPND susceptible cell state, and/or a normal or non-diseased cell state (e.g., a plurality of a cells of interest from a subject can be sequenced and a distribution is determined for the expression of genes, gene expression cassettes, or gene expression signatures). In certain embodiments, the distribution is a count-based metric for the number of transcripts of each gene present in a cell. A statistical difference between the distributions indicates a shift. The one or more genes, gene expression cassettes, or gene expression signatures can be selected to compare transcriptional identity based on the one or more genes, gene expression cassettes, or gene expression signatures having the most variance as determined by methods of dimension reduction (e.g., tSNE analysis).

In some embodiments, statistical shifts can be determined by defining a normal or non-diseased cell state, an AHPND tolerant cell state, and/or an AHPND susceptible cell state score. For example, a gene list of key genes enriched in an AHPND tolerant/susceptible model can be defined. To determine the fractional contribution to a cell's transcriptome to that gene list, the total log (scaled UMI+1) expression values for gene with the list of interest are summed and then divided by the total amount of scaled UMI detected in that cell giving a proportion of a cell's transcriptome dedicated to producing those genes. Thus, statistically significant shifts can be shifts in an initial score for the AHPND tolerant cell state score towards the AHPND susceptible cell state score and vice versa.

The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein can refer to a single mRNA or target nucleic acid to be sequenced. The UMI can also be used to determine the number of transcripts that gave rise to an amplified product, or in the case of target barcodes as described herein, the number of binding events. In preferred embodiments, the amplification is by PCR or multiple displacement amplification (MDA). Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes can be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support. Design and construction of UMIs are generally known in the art and can be used with the methods herein. See e.g., Islam S. et al., 2014. Nature Methods No: 11, 163-166, International Patent Publication No. WO 2014/047561. Other barcoding and tagging methods can be used with the invention herein, which are also known in the art. See e.g. Kress et al., “Use of DNA barcodes to identify flowering plants” Proc. Natl. Acad. Sci. U.S.A. 102(23):8369-8374 (2005), Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009), CBOL Plant Working Group, “A DNA barcode for land plants” PNAS 106(31):12794-12797 (2009), Kress et al., “DNA barcodes: Genes, genomics, and bioinformatics” PNAS 105(8):2761-2762 (2008), Lahaye et al., “DNA barcoding the floras of biodiversity hotspots” Proc Natl Acad Sci USA 105(8):2923-2928 (2008), Ausubel, J., “A botanical macroscope” Proceedings of the National Academy of Sciences 106(31):12569 (2009), Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

In some embodiments, the method can include generating a sequencing library. Methods of generating such a library are generally known in the art and can be used with the invention described herein.

Other methods for assessing differences in the AHPND tolerant cells/organisms and AHPND susceptible cells and/or organisms can be employed. In certain example embodiments, an assessment of differences in the AHPND tolerant and the AHPND susceptible proteome can be used to further identify key differences in cell type and sub-types or cells. states. For example, isobaric mass tag labeling and liquid chromatography mass spectroscopy can be used to determine relative protein abundances in the ex vivo and in vivo systems. Description provided elsewhere herein provides further disclosure on leveraging proteome analysis within the context of the methods disclosed herein.

The methods described herein can be used to identify and isolate cells and/or non-human organisms that are tolerant and/or susceptible to AHPND. The identified non-human organisms, such as crustaceans, can be used to identify lineages that can be used to improve the tolerance of an organism to AHPND. For example, lineages of non-human organisms, such as crustaceans, tolerant to AHPND can be used in breeding programs to raise generations of non-human organisms that can have improved AHPND tolerance expression signatures, which can be used in animal production, such as aquaculture. In some embodiments, the identified non-human organisms can be used in aquaculture production. In some embodiments, the identified non-human organisms can be used in as parents in a breeding program for aquaculture production.

### Methods of Screening for Agents Capable of Increasing Tolerance to AHPND

Described herein are methods of screening for agents capable of increasing tolerance to AHPND in an organism. The organism can be an organism that is susceptible to AHPND. The organism can be an organism that is tolerant to AHPND, where the agent can further increase the tolerance of the organism to AHPND.

In some embodiments, the method can include exposing and/or contacting a an AHPND tolerant cell and/or AHPND susceptible cell with one or more test agents and determining, detecting and/or measuring the presence and/or amount of a change in the phenotype, characteristic, signature, and/or activity, function and/or expression of one or more of the biomarkers as set forth in Table 1. A detected and/or measured change can be positive or negative (i.e., can be beneficial or detrimental). A detected and/or measured change can be an increase in a measured value or decrease in a measured value. A detected and/or measured change can be indicative of increased tolerance or susceptibility to AHPND. A detected and/or measured change can be indicative of decreased tolerance or susceptibility to AHPND. Methods of detecting and/and or measuring a change in phenotype, characteristic, signature, and/or activity, function and/or expression of one or more of the biomarkers in Table 1 are described in greater detail elsewhere herein (see e.g., section titled “METHODS OF DETECTING ORGANISMS SUSCEPTIBLE AND/OR TOLERANT TO AHPND” and Working Examples elsewhere herein).

Described in certain example embodiments herein are methods of screening for an agent effective to modify the acute hepatopancreatic necrosis disease (AHPND) tolerance of an organism and/or modify the AHPND susceptibility of an organism, including contacting an AHPND tolerant cell or an AHPND susceptible cell having an initial cell signature and/or cell state or type with a test agent; and determining a change in the initial cell signature, a shift in initial cell state or type, or both, wherein a change in the initial cell signature, a shift in initial cell state or type or both identifies, the test agent as an agent effective to modify the AHPND tolerance or susceptibility of the cell, organism, or both, and wherein determining a change in the initial cell signature, a shift in initial cell state or type, or both is composed of a method as previously described in any one of paragraphs [0007]-[0014] and elsewhere herein.

Agents identified as capable of increasing the tolerance to AHPND can, in some embodiments, increase AHPND tolerance 1-1000 fold or more, such as 0, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 fold or more. Agents identified as capable of decreasing the susceptibility to AHPND can, in some embodiments, decrease AHPND susceptibility 1-1000 fold or more, such as 0, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 fold or more.

Agents that increase tolerance and/or reduce susceptibility to AHPND as based on the assays and methods described herein can be identified as agents that increase tolerance to AHPND. Such agents can be administered to an organism or population thereof. In some embodiments, this administration of agents that increase the tolerance of and/or reduce susceptibility to AHPND can be administered in conjunction with one or more current methods of AHPND control, treatment, and/or mitigation.

As used herein, “agent” refers to any substance, compound, molecule, environmental condition(s) and the like, which can be biologically active or otherwise can induce a biological and/or physiological effect on and/or in a subject to which it is administered to or in which a subject is exposed to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Methods of Treating and Preventing AHPND Infection

Described herein are methods of treating and/or preventing AHPND infection that can include detecting AHPND tolerant and/or susceptible non-human organisms, such as crustaceans, in a population thereof and administering a suitable treatment and/or preventative based on detection of AHPND tolerant and/or susceptible non-human organisms. Methods of detecting and/and or measuring a change in phenotype, characteristic, signature, and/or activity, function and/or expression of one or more of the biomarkers in Table 1 are described in greater detail elsewhere herein (see e.g., section titled “METHODS OF DETECTING ORGANISMS SUSCEPTIBLE AND/OR TOLERANT TO AHPND” and Working Examples elsewhere herein). Suitable treatments and/or preventatives can include, but are not limited to, to those compositions, methods, and techniques currently used to manage, mitigate, control, or treat AHPND. It will be appreciated that the signatures and methods described herein can allow for a different and more precise timing and/or amount of a treatment/prevention to be applied, even if such methods are currently used. In other words, the signatures and methods described herein can change how currently treatments and/or preventions are used and the methods by which they are used. The methods and signatures described herein can, in some embodiments, result in less and/or more efficient use of treatments and preventatives, which can lead to reduced toxicity, waste, and/or costs.

In some embodiments, a treatment and/or preventative is administered to an AHPND susceptible subject or subject population 1, 2, 3, 4, 5, 6, 7, 8, 9, 10, 11, 12, or more days or months sooner than an AHPND tolerant subject or subject population. In some embodiments, the amount of a treatment and/or preventative used is reduced by 10, 20, 30, 40, 50, 60, 70, 80, 90, 100, 125, 150, 175, or 200 percent or more for an AHPND tolerant subject or subject population as compared to an AHPND susceptible subject or subject population.

Described in certain example embodiments herein are methods of treating or preventing acute hepatopancreatic necrosis disease (AHPND) in an organism or population thereof including detecting an AHPND tolerant organism and/or AHPND susceptible organism as previously described in any one of paragraphs [0007]-[0014] and elsewhere herein and
(a) administering an effective amount of an agent effective to treat or prevent AHPND infection;
(b) administering an effective amount of an agent effective to increase the tolerance of the organism to AHPND and/or reduce the susceptibility of the organism to AHPND;
(c) perform techniques capable of reducing AHPND in the environment;
(d) perform techniques capable of decreasing the mortality of AHPND in the organism(s);
(e) perform techniques capable of reducing the spread of and/or introduction of AHPND into the organism or population thereof; or
(f) any combination thereof.
   Modified Cells and Organisms with Tolerance to AHPND

Described in certain example embodiments herein are cells, cell populations, or progeny thereof including an acute hepatopancreatic necrosis disease (AHPND) signature, wherein the AHPND signature is composed of
(a) one or more genes selected from the group consisting of: SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of: PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof
(e) one or more genes selected from the group consisting of: CLT1-like, SP, CHYB, CRST-P, PPEA2, and CHYA;
(e) LGBP;
(f) one or more genes selected from the group consisting of: CRST-P, SP, PPEA2, CHYA, and LGBP;
(g) one or more genes selected from the group consisting of: SP, CRST-P, PPAE2, CHYA; or
(h) a combination thereof.

In certain example embodiments, the cell is a crustacean cell.

In certain example embodiments, the crustacean is a crab, lobster, crayfish, shrimp, prawn, or krill.

In certain example embodiments, the AHPND signature is composed of
(a) decreased expression of SP as compared to a suitable control and increased expression of CHYA, LGBP, and PPEA2 as compared to a suitable control;
(b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal;
(c) increased expression of PPAE2 and CHYA as compared to a suitable control and decreased expression of CTL1-like, CRST-P, SP, and CHYB as compared to a suitable control;
(d) increased expression of CRST-P, PPAE2, and CHYA as compared to a control sample obtained from an AHPND tolerant animal;
(e) decreased expression of LGBP as compared to a suitable control;
(f) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal;
(g) increased expression of PPEA2, CHYA, and LGBP as compared to a suitable control and decreased expression of CRST-P and SP as compared to a suitable control;
(h) increased expression of SP as compared to a control sample obtained from an AHPND susceptible animal and decreased expression of CRST-P, PPEA2, and CHYA as compared to a control sample obtained from an AHPND susceptible animal; or
(i) a combination thereof.

In certain example embodiments, the AHPND signature composed of (a), (b), (c), (d), or a combination thereof indicates that the organism or cell(s) thereof is susceptible to AHPND or that the sample contains at least one cell that is susceptible to AHPND.

In certain example embodiments, the AHPND signature composed of (e), (f), (g), (h), or a combination thereof indicates that the organism or cell(s) thereof is tolerant to AHPND or that the sample contains at least one cell that is tolerant to AHPND.

Also described herein are modified non-human organisms, such as crustaceans, that can be modified to have an increased tolerance of and/or reduced susceptibility to AHPND. In some embodiments, the modified non-human organism can have modulated expression and/or activity of one or more genes as set forth in Table 1 and/or a gene product(s) thereof. In some embodiments, the modified non-human organism can have modulated expression and/or activity of one or more of SEP8, BGBP, CRST-P CTL1-like, KPI, LGBP, EC-SOD, SP, PEN2, PPAE2, CHYA, CHYB, and any combination thereof. In some embodiments, expression and/or activity of the one or more gene products is increased in the modified non-human organism. In some embodiments, expression and/or activity of the one or more gene products is decreased in the non-human organism.

Described in certain example embodiments herein are modified non-human organism including one or more modified genes or expression thereof, wherein the one or more genes are one or more genes of an AHPND signature and are
(a) one or more genes selected from the group consisting of SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of: PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof;
(e) one or more genes selected from the group consisting of: CLT1-like, SP, CHYB, CRST-P, PPEA2, and CHYA;
(e) LGBP;
(f) one or more genes selected from the group consisting of: CRST-P, SP, PPEA2, CHYA, and LGBP;
(g) one or more genes selected from the group consisting of: SP, CRST-P, PPAE2, CHYA; or
(h) a combination thereof.

In certain example embodiments, the modified non-human organism is modified to include an AHPND tolerant signature.

In certain example embodiments, the AHPND tolerant signature is composed of
(a) decreased expression of LGBP as compared to a suitable control;
(b) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal;
(c) increased expression of PPEA2, CHYA, and LGBP as compared to a suitable control and decreased expression of CRST-P and SP as compared to a suitable control;
(d) increased expression of SP as compared to a control sample obtained from an AHPND susceptible animal and decreased expression of CRST-P, PPEA2, and CHYA as compared to a control sample obtained from an AHPND susceptible animal; or
(e) a combination thereof.

In certain example embodiments, the modified non-human organism is a crustacean.

In certain example embodiments, the crustacean is a crab, lobster, crayfish, shrimp, prawn, or krill.

In some embodiments, the modified non-human organism is modified to have decreased expression and/or activity of LGPB, PPAE2, CHYA, CRST-P, CTL1-like, and any combination thereof as compared to a suitable control. In some embodiments, the control is a sample prepared from an AHPND susceptible organism.

In some embodiments, the non-human modified organism is modified such that it has increased expression and/or activity of CHYB, SP, and any combination thereof as compared to a suitable control, where the suitable control is a sample prepared from an AHPND susceptible organism.

In some embodiments, the modified non-human organism is modified such that it has a) decreased expression of LGPB, PPAE2, CHYA, CRST-P, CTL1-like, and any combination thereof; b) increased expression of CHYB, SP, and any combination thereof; or c) both, where the suitable control is a sample prepared from an AHPND susceptible organism.

In some embodiments, the modified non-human organism is a non-human organism that can be infected by the causative agent(s) of AHPND. In some embodiments, the modified organism is a crustacean. In some embodiments, the modified non-human organism is a shrimp.

Methods and techniques of genetically modifying crustaceans, such as shrimp, are generally known in the art (see e.g., Beardmore and Porter. 2002. FAO Fisheries Circular no. 989; Yang et al. 2016. March Drugs. 14(8) pii: E152. doi: 10.3390/md14080152; Chen at al. 2019. March Biotechnol. February; 21(1):9-18. doi: 10.1007/s10126-018-9862-0; Li and Tsai. 2000. Mol. Reprod Dev. 56(2):149-154; Zhang et al., 2018. Fish Shellfish Immunol. 77:244-251. doi: 10.1016/j.fsi.2018.04.002; Gui et al. 2016. November 8; 6(11):3757-3764. doi: 10.1534/g3.116.034082, the teachings of which can be adapted for use in the present invention.

Methods can include, but are not limited to, traditional knock in and knock down approaches based on homologues recombination, as well as more recent nuclease-based approaches such as zinc-finger nucleases, transcription activator-like effector nucleases, and CRISPR-Cas systems. Such systems and techniques are known in the art and can be adapted for use with the present invention.

Also described herein are polynucleotides and vectors that can be used to generate such modified organisms.

### Vectors

Also provided herein are vectors that can contain one or more of the polynucleotides capable of generating a modified non-human organism described elsewhere herein. The vectors can be useful in producing bacterial, fungal, yeast, plant cells, animal cells, and transgenic animals that can express and/or modify the expression of one or more genes of Table 1. Within the scope of this disclosure are vectors containing one or more of the polynucleotide sequences described herein. One or more of the polynucleotides described herein can be included in a vector or vector system. The vectors and/or vector systems can be used, for example, to express one or more of the polynucleotides in a cell, such as a producer cell, to produce viral particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment can be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that include one or more free ends, no free ends (e.g., circular); nucleic acid molecules that include DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for expression and/or delivery of a polynucleotide described herein. In some embodiments, expression of a polynucleotide described herein can be driven by the CBh promoter. Where the element of the polynucleotide is an RNA, its expression can be driven by a Pol III promoter, such as a U6 promoter. In some embodiments, the two are combined.

### Cell-Based Vector Amplification and Expression

Vectors can be designed for expression of one or more polynucleotides described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, crustacean cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U20S, A549, HT1080, CAD, P19, NIH 3T3, L929, N2a, MCF-7, Y79, SO-Rb50, HepG G2, DIKX-X11, J558 L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (see e.g., Baldari, et al., 1987. EMBO J. 6: 229-234), pMFa (see e.g., Kuijan and Herskowitz, 1982. Cell 30: 933-943), pJRY88 (see e.g., Schultz et al., 1987. Gene 54: 113-123), pYES2 (see e.g., Invitrogen Corporation, San Diego, Calif.), and picZ (see e.g., InVitrogen Corp, San Diego, Calif.). As used herein, a “yeast expression vector” refers to a nucleic acid that contains one or more sequences encoding an RNA and/or polypeptide and can further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in e.g., Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast can include plasmids, yeast artificial chromosomes, 2p plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (see e.g., Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (see e.g., Seed, 1987. Nature 329: 840) and pMT2PC (see e.g., Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; see e.g., Pinkert, et al., 1987. Genes Dev. 1: 268-277), lymphoid-specific promoters (Calame and Eaton, 1988. Adv. Immunol. 43: 235-275), in particular promoters of T cell receptors (see e.g., Winoto and Baltimore, 1989. EMBO J. 8: 729-733) and immunoglobulins (see e.g., Baneiji, et al., 1983. Cell 33: 729-740; Queen and Baltimore, 1983. Cell 33: 741-748), neuron-specific promoters (see e.g., the neurofilament promoter; Byrne and Ruddle, 1989. Proc. Natl. Acad. Sci. USA 86: 5473-5477), pancreas-specific promoters (see e.g., Edlund, et al., 1985. Science 230: 912-916), and mammary gland-specific promoters (see e.g., milk whey promoter; U.S. Pat. No. 4,873,316 and European Application Publication No. 264,166). Developmentally-regulated promoters are also encompassed, e.g., the murine hox promoters (see e.g., Kessel and Gruss, 1990. Science 249: 374-379) and the α-fetoprotein promoter (see e.g., Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more polynucleotides described herein so as to drive expression of the one or more elements of the polynucleotides described herein described herein.

Vectors can be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (see e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (see e.g., Pharmacia Biotech Inc; Smith and Johnson, 1988. Gene 67: 31-40), pMAL (see e.g., New England Biolabs, Beverly, Mass.) and pRIT5 (see e.g., Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (see e.g., Amrann et al., (1988) Gene 69:301-315) and pET 11d (see e.g., Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more polynucleotides described herein are introduced into a host cell such that expression of the polynucleotide(s) described herein can result in modifying the genome, transcriptome, proteome, and/or epigenome of a non-human organism. For example, different polynucleotides described herein can each be operably linked to separate regulatory elements on separate vectors. RNA(s) of present invention described herein can be delivered to an animal or mammal or cell thereof to produce an animal or mammal or cell thereof that constitutively or inducibly or conditionally expresses different polynucleotides described herein that incorporates one or more polynucleotides described herein or contains one or more cells that incorporates and/or expresses one or more polynucleotides described herein.

In some embodiments, two or more of the elements expressed from the same or different regulatory element(s), can be combined in a single vector, with one or more additional vectors providing any components of the system not included in the first vector. Polynucleotides described herein that are combined in a single vector can be arranged in any suitable orientation, such as one element located 5′ with respect to (“upstream” of) or 3′ with respect to (“downstream” of) a second element. The coding sequence of one element can be located on the same or opposite strand of the coding sequence of a second element, and oriented in the same or opposite direction. In some embodiments, a single promoter drives expression of a polynucleotides described herein, embedded within one or more intron sequences (e.g., each in a different intron, two or more in at least one intron, or all in a single intron). In some embodiments, the polynucleotides described herein can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (see e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In embodiments, the polynucleotides and/or vectors thereof described herein (such as the polynucleotides of the present invention) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements can also direct expression in a temporal-dependent manner, such as in a cell-cycle dependent or developmental stage-dependent manner, which may or may not also be tissue or cell-type specific. In some embodiments, a vector includes one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (see e.g., Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter can be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g., Myh6 (alpha MHC), MYL2 (MLC-2v), TNI3 (cTn1), NPPA (ANF), Slc8a1 (Ncx1)), central nervous system cell promoters (e.g., SYN1, GFAP, INA, NES, MOBP, MBP, TH, FOXA2 (HNF3 beta)), skin cell specific promoters (e.g., FLG, K14, TGM3), immune cell specific promoters, (e.g., ITGAM, CD43 promoter, CD14 promoter, CD45 promoter, CD68 promoter), urogenital cell specific promoters (e.g., Pbsn, Upk2, Sbp, Fer1l4), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, A1cA, LexA, Hsp70 promoter, Hsp90 promoter, pDawn, XVE/OlexA, GVG, and pOp/LhGR.

Where expression in a plant cell is desired, the polynucleotides described herein described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters can direct the expression of a gene in different tissues or cell types, or at different stages of development, or in response to different environmental conditions. In particular embodiments, one or more of the polynucleotides described herein are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the expression of polynucleotides described herein are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression can use a form of energy. The form of energy can include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system can include one or more polynucleotides described herein, a light-responsive cytochrome heterodimer (e.g., from Arabidopsis thaliana), and a transcriptional activation/repression domain. In some embodiments, the vector can include one or more of the inducible DNA binding proteins provided in PCT publication WO 2014/018423 and U.S. Publications, 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the vector or system thereof can include one or more elements capable of translocating and/or expressing a polynucleotides described herein to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the polynucleotides described herein can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in a polynucleotide described herein such that the selectable marker polypeptide, when translated, is inserted between two amino acids between the N- and C-terminus of the polynucleotides described herein or at the N- and/or C-terminus of the polynucleotides described herein. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the polynucleotides described herein described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more polynucleotides described herein described herein via suitable linker, such as a glycine or glycine serine linkers as short as GS or GG up to (GGGGG)₃ (SEQ ID NO: 1) or (GGGGS)₃ (SEQ ID NO: 2). Other suitable linkers are described elsewhere herein and/or generally known in the art.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the polynucleotides described herein and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated polynucleotides described herein to specific cells, tissues, organs, etc.

### Cell-Free Vector and Polynucleotide Expression

In some embodiments, the polynucleotide encoding one or more polynucleotides described herein can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding one or more polynucleotides described herein can be codon optimized. In some embodiments, one or more polynucleotides contained in a vector (“vector polynucleotides”) described herein that are in addition to an optionally codon optimized polynucleotide described herein. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, P A), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gown, Plant Physiol. 1990 January; 92(1): 1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal, avian, or crustacean) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, hepatopancreatic cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, hepatopancreas, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells can be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with one or more polynucleotides described herein of the present invention and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as one or more polynucleotides described herein of the present invention.

### Naked Polynucleotides

In some embodiments, one or more polynucleotides described herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the polynucleotides described herein of the present invention can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the polynucleotide(s) described herein of the present invention. In some embodiments, the naked polynucleotide can contain other nucleic acids and/or polynucleotides in addition to the polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the polynucleotides described herein of the present invention can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments, a non-viral polynucleotide transposon vector system can include a first polynucleotide vector that contains the polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g. the polynucleotide(s) of the present invention) and integrate it into one or more positions in the host cell's genome. In some embodiments, the transposon vector or system thereof can be configured as a gene trap. In some embodiments, the TIRs can be configured to flank a strong splice acceptor site followed by a reporter and/or other gene (e.g., one or more of the polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, Sleeping Beauty transposon system (Tcl/mariner superfamily) (see e.g. Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof.

### Chemical Carriers

In some embodiments, the polynucleotide(s) described herein (such as those that can be used to generate a modified non-human organism) can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the polynucleotide(s) of the present invention), (2) those capable of targeting specific cells, (3) those capable of increasing delivery of the polynucleotide (such as the polynucleotide(s) of the present invention) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the polynucleotides described herein of the present invention. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticulo endothelial system. In some embodiments, the inorganic particles can be optimized to protect an entrapped molecule from degradation, the Suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., superparamagnetic iron oxide and magnetite), quantum dots, fullerenes (e.g. carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g., such as the polynucleotide(s) of the present invention). In some embodiments, chemical non-viral carrier systems can include a polynucleotide such as the polynucleotide(s) of the present invention) and a lipid (such as a cationic lipid). These are also referred to in the art as lipoplexes. Other embodiments of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g., the polynucleotide(s) of the present invention). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g., polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., U.S. Pat. Pub. 2017/0079916 whose techniques and compositions can be adapted for use with the polynucleotide(s) of the present invention), polymethacrylate, and combinations thereof.

In some embodiments, the non-viral carrier can be configured to release a polynucleotide of the present invention that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the polynucleotide(s) of the present invention described herein and an environmental triggering agent response element, and optionally a triggering agent. In some embodiments, the particle can include a polymer that can be selected from the group of polymethacrylates and polyacrylates. In some embodiments, the non-viral particle can include one or more embodiments of the compositions microparticles described in U.S. patentt. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the polynucleotide(s) of the present invention). Polymer-based systems are described in greater detail elsewhere herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as the polynucleotide(s) of the present invention, into a virus particle and producing said virus particle when used alone or with one or more other viral vectors (such as in a viral vector system). Viral vectors and systems thereof can be used for producing viral particles for delivery of and/or expression of one or more polynucleotides of the present invention described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system can therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the polynucleotides of the present invention described herein.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g., promoter(s)) to drive expression of the features present on the packaging vector, and (iii) an “envelope vector” that contains one or more envelope protein genes and upstream promoters. In embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see e.g., DiGiusto et al. (2010) Sci Transl Med 2:36ra43) can be used/and or adapted to the polynucleotide(s) of the present invention.

In some embodiments, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically include proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA) (SEQ ID NO: 3) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA (SEQ ID NO: 3) can be fused to a cell targeting peptide and the TEFCA-CPT fusion construct can be incorporated into the same or a different lentiviral vector as the PDZ1-envelope protein construct. During virus production, specific interaction between the PDZ1 and TEFCA (SEQ ID NO: 3) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., U.S. Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., U.S. Patent Publication Nos. US20110293571; US20110293571, US20040013648, US20070025970, US20090111106 and U.S. Pat. No. 7,259,015. Any of these systems or a variant thereof can be used to deliver one or more inventive polynucleotides described herein to a cell.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2 or serotype 5. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In embodiments of the helper-dependent adenoviral vector system one vector (the helper) can contain all the viral genes required for replication but contains a conditional gene defect in the packaging domain. The second vector of the system can contain only the ends of the viral genome, one or more polynucleotides of the present disclosure described herein, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the one or more polynucleotides of the present disclosure. In some embodiments, the polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in the polynucleotide(s) of the present disclosure. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use with the polynucleotide(s) of the present disclosure. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use with the polynucleotide(s) of the present disclosure.

### Adeno Associated Viral (AAV) Vectors

In an embodiment, the vector can be an adeno-associated virus (AAV) vector. See e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E4ORF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008).

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the polynucleotide(s) of the present disclosure).

### Herpes Simplex Viral Vectors

In some embodiments, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use with the polynucleotide(s) of the present disclosure. In some embodiments where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiments the polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22 L10302-10312; and Betchen and Kaplitt. 2003. Curr. Opin. Neurol. 16:487-493, whose techniques and vectors described therein can be modified and adapted for use with the polynucleotide(s) of the present disclosure.

### Poxvirus Vectors

In some embodiments, the vector can be a poxvirus vector or system thereof. In some embodiments, the poxvirus vector can result in cytoplasmic expression of one or more polynucleotides of the present disclosure. In some embodiments, the capacity of a poxvirus vector or system thereof can be about 25 kb or more. In some embodiments, a poxvirus vector or system thereof includes one or more polynucleotide(s) of the present disclosure.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. application publication No. U.S. 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more polynucleotide(s) of the present disclosure described herein are as used in the foregoing documents, such as WO 2014/093622 (PCT/US2013/074667) and are discussed in greater detail herein.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., the inventive polynucleotide(s)), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered inventive polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the inventive polynucleotide(s) described herein) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., transcripts, proteins, enzymes, mutant forms thereof, fusion proteins thereof, etc.), and virus particles (such as from viral vectors and systems thereof).

One or more polynucleotides of the present disclosure can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV can be advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of the polynucleotides of the present disclosure can be delivered to cells via particles. The term “particle” as used herein, refers to any suitable sized particles for delivery of the polynucleotide(s) of the present disclosure. Suitable sizes include macro-, micro-, and nano-sized particles. In some embodiments, any of the of the polypeptides, polynucleotides, vectors and combinations thereof described herein) can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in embodiments, particle delivery can also be advantageous for other molecules and formulations described elsewhere herein.

Further embodiments are illustrated in the following Examples which are given for illustrative purposes only and are not intended to limit the scope of the disclosure.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Differentially Expressed Genes in Hepatopancreas of AHPND Tolerant and Susceptible Shrimp (Penaeus vannamei)

### Materials and Methods

Bioassay 1-Assessing AHPND Tolerance in P. vannamei

Vibrio parahaemolyticus (Strain 13-028A/3) (Vp_{AHPND}) was used for the experimental challenges following a previously published protocol¹⁵. Three P. vannamei family lines were obtained from a commercial supplier as a part of an on-going family line screening for AHPND-tolerance. Each line was stocked separately into a total of nine 1000 L tanks in triplicate. From each of these three lines, we screened 56, 57 and 78 organisms from lines one, two and three respectively. A Specific Pathogen Free (SPF) P. vannamei (N=60, average weight 3 g) (AHPND susceptible line, population P1, were obtained from a commercial supplier in the USA and stocked into two 90 L tanks and used as positive controls for AHPND challenge. A third tank containing SPF shrimps of the same genetic line (N=64) was used as negative control. An immersion challenge was performed using an inoculum load of 10⁶ cfu/ml¹⁵. The experiment was terminated at 7 days post-inoculation. The mortality in each tank was recorded daily, and a subset of moribund and surviving animals was examined by routine H&E histology.

### Histopathology

Moribund P. vannamei were fixed in Davidson's alcohol-formalin-acetic acid (AFA) fixative. The samples were processed, embedded in paraffin, sectioned (4 μm thick) and analyzed in accordance with standard methods.

### Bioassay 2—Initial Gene Expression Analyses

To evaluate gene expression in the AHPND-tolerant and -susceptible lines, a second challenge was conducted. A total of 46 animals were utilized from P1 (n=42, average weight 5 g) with 21 animals challenged with VP_{AHPND}, 21 animals utilized as negative controls and 4 animals sampled for gene expression analysis prior to challenge. The tolerant line was named population P2 (n=20, average weight 9 g), with 9 animals challenged with VP_{AHPND}, 9 unchallenged as negative controls and 2 animals sampled for gene expression analysis prior to challenge. All shrimp from each family were tagged in the 4th abdominal segment with uniquely colored elastomer tags to allow visual identification. Both families were then held in a single 1000 L tank and challenged by immersion, as described above. The negative controls were held in a single 1000 L tank but were not challenged.

Twenty-four hours after challenge, animals were collected for gene expression analysis. Limited numbers (N=4) of the susceptible P1 line were available for sampling due to the typically high mortality rates observed in AHPND challenges in the first 24 hours. Two shrimp were collected from the tolerant population P2 at 24 hours post-infection to leave enough animals in the tank for a survival comparison at termination. In order to keep the sample numbers equal, a pool of 10 challenged animals from P2 group in the initial family line challenge were pooled as 2 samples for a total of 4 samples.

Measuring the Expression Levels of Candidate Immune and Metabolic Genes in AHPND Challenged P. vannamei

Four shrimp samples per population were collected from each treatment for RNA extraction using RNAzol following the manufacturer's protocol (MRC, Ohio, USA). The RNA was treated with DNase 1 (Invitrogen, USA). One μg of DNase treated RNA was used for cDNA synthesis using Superscript IV and following the manufacturer's recommendation (Invitrogen, USA). After that, cDNA was subjected to gene expression analysis.

The mRNA expression of β-glucan binding protein (BGBP) (AY249858.1), Crustin-P (CRSTP) (AY488497), C-type lectin 1-like (CTL1-like) (DQ858900.1), Extracellular Copper/Zinc Superoxide dismutase (EC-SOD) (HM371157), Kazal protease inhibitor (KPI) (AY544986), lipopolysaccharide and β-1,3-glucan-binding protein (LGBP) (EU102286.1), Penaidin 2 (PEN2) (Y14925), Prophenol oxidase activation system 2 (PPAE2) (AWF98992.1), Serpin8 (SEP8) (KU853046.1), serine protease (SP) (AY368151), Chymotrypsin A (ChyA) (Y10664), Chymotrypsin B (ChyB) (Y10665) and two toxin genes of V. parahaemolyticus (i.e. pirA and pirB toxin genes, KM067908) were measured by quantitative PCR (StepOnePlus Real-time PCR system, Applied biosystems, USA) using PowerUP™ SYBR Green Master Mix (Applied Biosystem, USA). The reaction mixture contained 10 μl of PowerUP™ SYBR Green Master Mix, 0.8 μl (0.4 μM) of each primer, 2.0 μl of cDNA and 6.4 μl of sterile water in a reaction volume of 20 μl. The thermal profile for the reaction was 2 minutes at 50° C., 2 minutes at 95° C. followed by 40 cycles of 3 seconds at 95° C. and 30 seconds at 60° C. The primer sequence for each gene is given in Table 1. Each sample was run in duplicate and the mean Ct value was used for gene expression analysis. Expression levels of each gene in various populations are shown relative to the expression in corresponding control treatments according to the formula of Livak and Schmittgen²⁵. For evaluating the gene expression between challenged animals coming from P1 and P2 population, the expression levels of each gene were shown relative to the expression in the P1 negative control. The expression value was converted to Log based 2 prior to statistical analysis. The genes showing statistical difference in expression level in Bioassay 2 were validated in Bioassay 3.

TABLE 1
Genes Evaluated

| | | | | SEQ |
|---|---|---|---|---|
| | | Sequence | Refer- | ID |
| Genes | Primers | (5′ to 3′) | ences | NO: |
| SEP8 | Forward | CCGTG | (59) | 4 |
| | | CATAT | | |
| | | CCGAA | | |
| | | TTACT | | |
| | | CAA | | |
| | Reverse | GCACC | | 5 |
| | | ATCAG | | |
| | | GGCGT | | |
| | | TTCTC | | |
| BGBP | Forward | GCTGG | *This | 6 |
| | | CTTAC | study | |
| | | GGCTA | | |
| | | CGACT | | |
| | | T | | |
| | Reverse | GGAGA | | 7 |
| | | AGAAT | | |
| | | GACGA | | |
| | | GTCGG | | |
| | | TCTA | | |
| CRST- | Forward | AGCGA | (60) | 8 |
| P | | CTGCA | | |
| | | GGTAT | | |
| | | TGGTG | | |
| | Reverse | TCGTT | | 9 |
| | | GGAAC | | |
| | | AGGTT | | |
| | | GTGG | | |
| CTL1- | Forward | CAAGA | * | 10 |
| like | | CCCAC | This | |
| | | AGCGG | study | |
| | | AGAGA | | |
| | Reverse | TAGAC | | 11 |
| | | GAGGG | | |
| | | CGAAG | | |
| | | GTTTC | | |
| KPI | Forward | AGGCA | * | 12 |
| | | GCTGT | This | |
| | | TTGCA | study | |
| | | CGAAT | | |
| | Reverse | AGTGC | | 13 |
| | | AGTCA | | |
| | | CAATT | | |
| | | GCCAT | | |
| | | CT | | |
| LGBP | Forward | CATGT | (34) | 14 |
| | | CCAAC | | |
| | | TTCGC | | |
| | | TTTCA | | |
| | | GA | | |
| | Reverse | ATCAC | | 15 |
| | | CGCGT | | |
| | | GGCAT | | |
| | | CTT | | |
| EC- | Forward | ATGAA | (61) | 16 |
| SOD | | GACGT | | |
| | | TGGCA | | |
| | | ACTCT | | |
| | | G | | |
| | Reverse | CTCGC | | 17 |
| | | AGGTG | | |
| | | GAGTG | | |
| | | GAG | | |
| SP | Forward | ACGTT | (60) | 18 |
| | | CTCAC | | |
| | | GACTG | | |
| | | GTCAC | | |
| | | AC | | |
| | Reverse | TATGT | | 19 |
| | | AAGGC | | |
| | | GCGTC | | |
| | | GTTCT | | |
| | | C | | |
| PEN2 | Forward | TCGTG | (60) | 20 |
| | | GTCTG | | |
| | | CCTGG | | |
| | | TCTT | | |
| | Reverse | CAGGT | | 21 |
| | | CTGAA | | |
| | | CGGTG | | |
| | | GTCTT | | |
| | | C | | |
| PPAE2 | Forward | TTCCT | (59) | 22 |
| | | TGGGT | | |
| | | GGCTG | | |
| | | CTTT | | |
| | Reverse | TGTTC | | 23 |
| | | GCCGA | | |
| | | GACGG | | |
| | | ATTAC | | |
| ChyA | Forward | AGCCA | * This | 24 |
| | | GCCAG | study | |
| | | GTCTC | | |
| | | CATT | | |
| | Reverse | AAGAG | | 25 |
| | | TTCCA | | |
| | | GTTCT | | |
| | | CGTGA | | |
| | | GTGA | | |
| ChyB | Forward | ACCGG | * This | 26 |
| | | CAGTA | study | |
| | | TCTCC | | |
| | | AACGT | | |
| | Reverse | TTGCA | | 27 |
| | | GTCGT | | |
| | | CGTTC | | |
| | | GTCAT | | |
| pirA | Forward | CAAAC | * This | 28 |
| | | GGAGG | study | |
| | | CGTCA | | |
| | | CAGA | | |
| | Reverse | GACCG | | 29 |
| | | ACTTC | | |
| | | CGGGA | | |
| | | TGAT | | |
| pirB | Forward | TGCAA | * This | 30 |
| | | ACCAA | study | |
| | | GATAA | | |
| | | CGTGT | | |
| | | ATGA | | |
| | Reverse | GCCGT | | 31 |
| | | GAACC | | |
| | | GTACA | | |
| | | CCAA | | |
| EFI- | Forward | TCGCC | (62) | 32 |
| α | | GAACT | | |
| | | GCTGA | | |
| | | CCAAG | | |
| | | A | | |
| | Reverse | CCGGC | | 33 |
| | | TTCCA | | |
| | | GTTCC | | |
| | | TTACC | | |

### Bioassay 3—Gene Expression Validation

Two hundred shrimp (average wt. 2 g) with the same genetic characteristics of the AHPND tolerant shrimp line (P2) from Bioassay 2 were stocked in two 1000 L-tanks (100 shrimp/tank) in which one tank was challenged with VP_{AHPND}. The remaining tank was used as negative control. Forty SPF shrimp (AHPND susceptible shrimp) (average wt. about 1.5 g) (P1) were stocked in two 90 L-tanks (20 shrimp/tank), one tank was challenged with VP_{AHPND}. The remaining tank was used as negative control. The AHPND challenge protocol was the same as described previously. After 24 hours, nine shrimp from each tank were collected for gene expression analyses.

### Statistical Analysis

The statistical significances in the difference in Log based 2 value of gene expression between control and challenged animals in each population and between challenged animals in P1 and P2 populations was determined by using Student's t-test with P<0.05, SPSS v.16 software. The mortality data was analyzed using Kaplan-Meier method, SPSS v.16 software.

### Results

Bioassay 1—Assessing AHPND Tolerance in P. vannamei

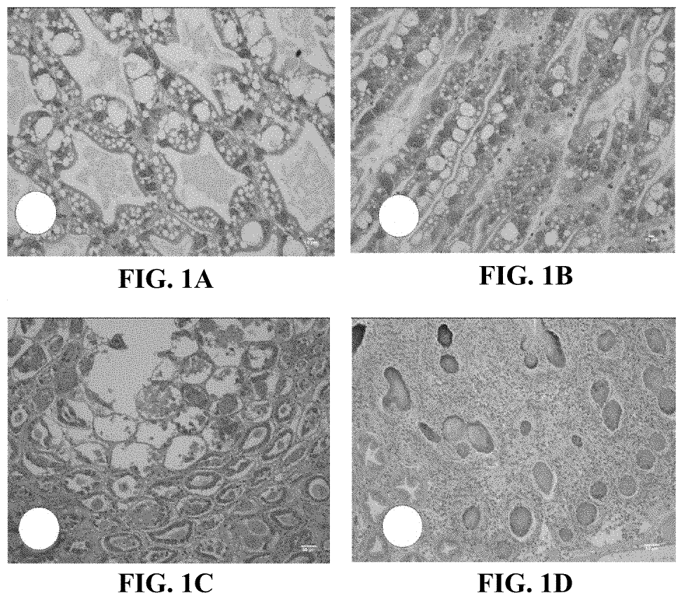

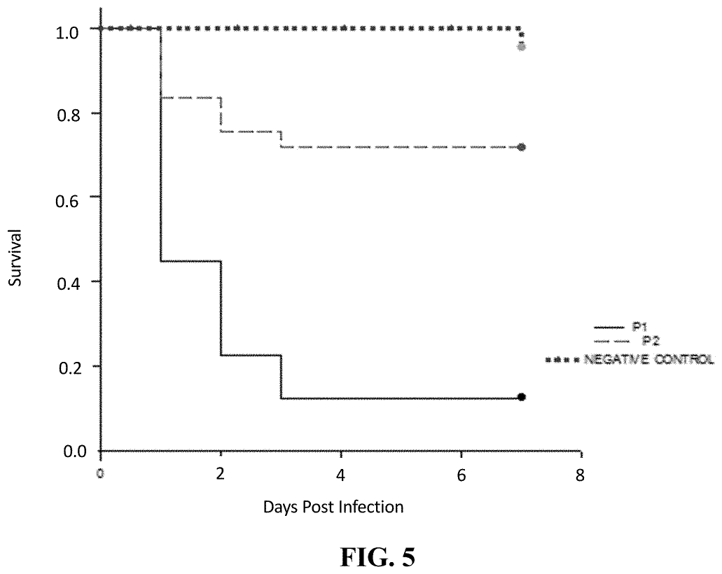

In experimental AHPND challenge, the average cumulative mortality in the AHPND-susceptible P1 population was three times higher compared to the AHPND-tolerant P2 population (87.5% vs. 28%). Meanwhile, the average cumulative mortality in negative control was 4.55% (FIG. 5). The histopathology of the P1 and P2 populations are presented in FIGS. 1A-1D. The Davidson-fixed shrimp from the healthy, negative controls from P1 and P2 displayed normal structure of tubules and epithelial cells in the hepatopancreas including high levels of lipid droplets (R-cells), secretory vacuoles (B-cells) and absence of AHPND (FIGS. 1A and 1B). In contrast, shrimp from the P1 population challenged with Vp_{AHPND} displayed lesions typical of AHPND in the acute phase, including a multifocal necrosis and massive sloughing of HP tubule epithelial cells in the hepatopancreas. At this stage, bacterial colonization was not observed (FIG. 1C). Shrimp from the P2 population displayed the typical VP_{AHPND} chronic phase characterized by SHPN-like lesions (FIG. 1D).

Bioassay 2—Initial Gene Expression Analyses, Measuring the mRNA Expression of Immune and Metabolic Genes in P. vannamei

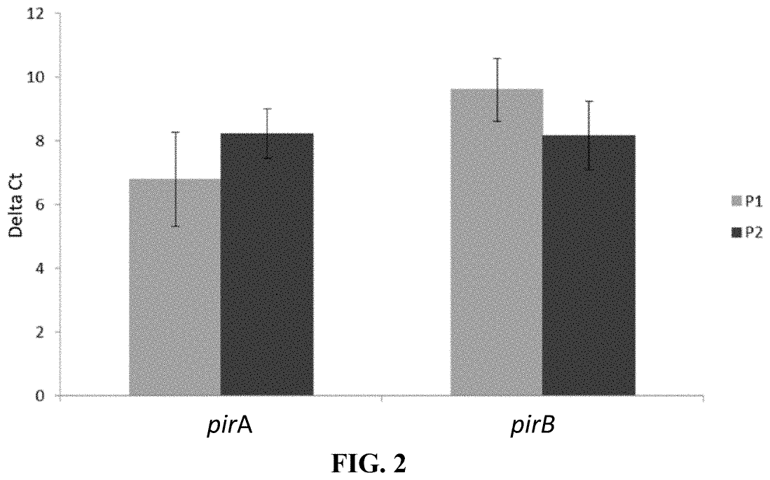

Interestingly, there was no significant difference in the expression levels of pirA/pirB genes between P1 and P2 population (P>0.05) (FIG. 2).

The mRNA expression of a set of immune and metabolic genes (i.e., SEP8, BGBP, CRSTP, CTL1-like, KPI, LGBP, EC-SOD, PEN2, PPAE2, SP, ChyA and ChyB) were measured by RT-qPCR in control animals and challenged animals in each population. We also compared the levels of gene expression in AHPND susceptible P1 and AHPND tolerant P2 populations.

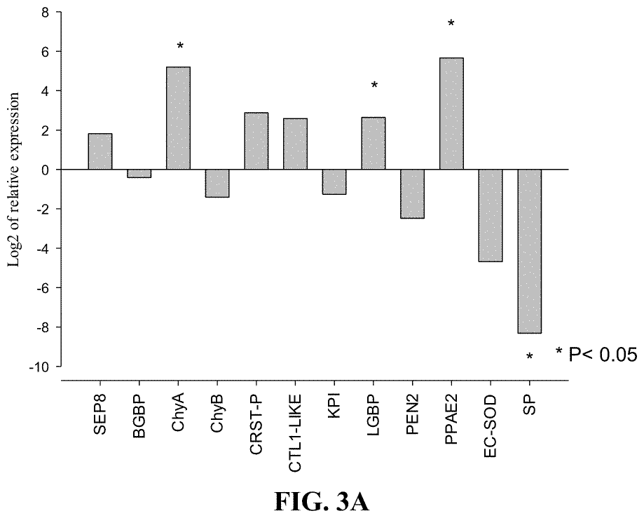

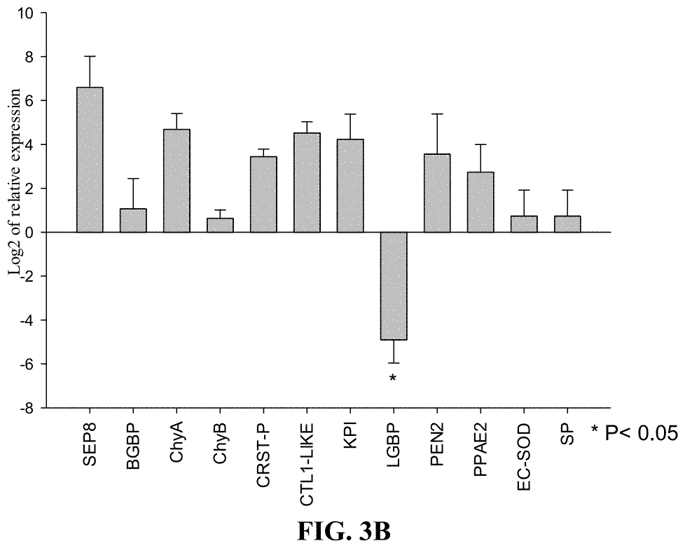

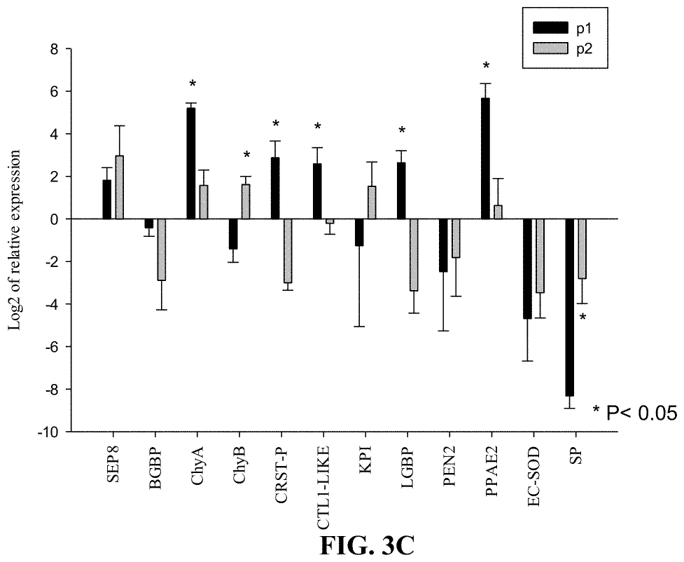

The V. parahaemolyticus infection led to the significant upregulation of expression of LGBP, PPAE2, and ChyA transcripts in the P1 population (P<0.05) whereas SP mRNA showed significant down regulated expression (P<0.05). The mRNA levels of BGBP, CRSTP, CTL1-like, KPI, PEN2, EC-SOD, SEP8 and ChyB in the challenged and un-challenged groups did not show significant differences (P>0.05) (FIG. 3A).

In the P2 population, V. parahaemolyticus infection led to the significant downregulated expression of LGBP mRNA (P<0.05). The expression of BGBP, SEP8, CTL1-like, CRSTP, KPI, EC-SOD, PPAE2, PEN2, SP, ChyA and ChyB showed no significant difference between challenged and un-challenged groups (P>0.05) (FIG. 3B)

When the mRNA expression levels were compared between AHPND challenged animals from the P1 and P2 populations, there were significant differences in the expression profiles of some genes. For example, while the susceptible P1 population showed significantly higher levels of expression of ChyA, CRSTP, CTL1-like, LGBP and PPAE2 (P<0.05) (FIG. 3C), the AHPND-tolerant population P2 showed higher expression of SP and ChyB compared to the susceptible P1 population (P<0.05) (FIG. 3C) (Table 2).

TABLE 2
Differentially expression of 12 immune and metabolic related genes between AHPND
susceptible (P1) and AHPND tolerant/resistant (P2) population (2^{nd} bio trial)

| | Before | After AHPND | | |
|---|---|---|---|---|
| | challenge-P1 | Challenge-P1 | | |
| Genes | (Avg. ΔCt) | (Avg. ΔCt) | Log₂2^{-ΔΔCt} | P-value |
| SEP | 10.23 ± 3.94 | 8.42 ± 1.38 | 1.81 ± 0.60 | 0.418 |
| BGBP | 6.38 ± 1.17 | 6.79 ± 0.77 | −0.41 ± 0.38 | 0.597 |
| ChyA | 2.01 ± 4.12 | −3.19 ± 0.57 | 5.20 ± 0.25 | 0.047* |
| ChyB | 8.23 ± 2.29 | 9.64 ± 1.47 | −1.41 ± 0.63 | 0.340 |
| CRST-P | 4.56 ± 4.29 | 1.68 ± 1.89 | 2.88 ± 0.99 | 0.281 |
| CTL-1-like | 3.79 ± 1.87 | 1.21 ± 1.30 | 2.58 ± 0.77 | 0.093 |
| KPI | 9.04 ± 3.57 | 10.30 ± 8.20 | −1.26 ± 3.80 | 0.804 |
| LGBP | 5.66 ± 0.60 | 3.02 ± 1.22 | 2.64 ± 0.59 | 0.012* |
| PEN2 | 8.80 ± 2.27 | 11.28 ± 6.36 | −2.48 ± 2.78 | 0.494 |
| PPAE2 | 10.30 ± 3.80 | 4.64 ± 1.62 | 5.67 ± 0.70 | 0.034* |
| SOD | 9.96 ± 3.78 | 14.64 ± 5.19 | −4.68 ± 2.25 | 0.827 |
| SP | 10.63 ± 3.79 | 18.95 ± 1.37 | −8.31 ± 0.59 | 0.006** |
| SEP | 10.23 ± 3.94 | 8.42 ± 1.38 | 1.81 ± 0.60 | 0.418 |
| BGBP | 6.38 ± 1.17 | 6.79 ± 0.77 | −0.41 ± 0.38 | 0.597 |
| ChyA | 2.01 ± 4.12 | −3.19 ± 0.57 | 5.20 ± 0.25 | 0.047* |
| ChyB | 8.23 ± 2.29 | 9.64 ± 1.47 | −1.41 ± 0.63 | 0.340 |
| CRST-P | 4.56 ± 4.29 | 1.68 ± 1.89 | 2.88 ± 0.99 | 0.281 |
| CTL-1-like | 3.79 ± 1.87 | 1.21 ± 1.30 | 2.58 ± 0.77 | 0.093 |
| KPI | 9.04 ± 3.57 | 10.30 ± 8.20 | −1.26 ± 3.80 | 0.804 |
| LGBP | 5.66 ± 0.60 | 3.02 ± 1.22 | 2.64 ± 0.59 | 0.012* |
| PEN2 | 8.80 ± 2.27 | 11.28 ± 6.36 | −2.48 ± 2.78 | 0.494 |
| PPAE2 | 10.30 ± 3.80 | 4.64 ± 1.62 | 5.67 ± 0.70 | 0.034* |
| SOD | 9.96 ± 3.78 | 14.64 ± 5.19 | −4.68 ± 2.25 | 0.827 |
| SP | 10.63 ± 3.79 | 18.95 ± 1.37 | −8.31 ± 0.59 | 0.006** |
| | Before | After AHPND | | |
| | challenge-P2 | Challenge-P2 | | |
| Genes | (Avg. ΔCt) | (Avg. ΔCt) | Log₂2^{-ΔΔCt} | P-value |
| SEP | 13.86 ± 1.16 | 7.26 ± 3.26 | 6.6 ± 1.41 | 0.057 |
| BGBP | 10.36 ± 5.24 | 9.29 ± 3.19 | 1.07 ± 1.38 | 0.763 |
| ChyA | 5.12 ± 11.2 | 0.43 ± 1.67 | 4.69 ± 0.72 | 0.403 |
| ChyB | 7.25 ± 0.33 | 6.62 ± 0.89 | 0.63 ± 0.39 | 0.278 |
| CRST-P | 11.00 ± 4.29 | 7.56 ± 0.80 | 3.44 ± 0.35 | 0.458 |
| CTL-1-like | 8.52 ± 7.81 | 4.00 ± 1.18 | 4.52 ± 0.51 | 0.563 |
| KPI | 11.74 ± 3.25 | 7.51 ± 2.66 | 4.23 ± 1.15 | 0.158 |
| LGBP | 4.13 ± 0.35 | 9.04 ± 2.48 | −4.91 ± 1.05 | 0.025* |
| PEN2 | 14.17 ± 0.18 | 10.61 ± 4.22 | 3.56 ± 1.83 | 0.324 |
| PPAE2 | 12.41 ± 0.90 | 9.67 ± 2.92 | 2.74 ± 1.26 | 0.286 |
| SOD | 14.17 ± 0.18 | 13.43 ± 2.74 | 0.74 ± 1.18 | 0.739 |
| SP | 14.17 ± 0.18 | 13.43 ± 2.74 | 0.74 ± 1.18 | 0.739 |

ΔCt was calculated as Ct_{(target gene)} − Ct_{(EF-1α)}
ΔΔCt was calculated as ΔCt_{(challenged} ₐₙᵢₘₐₗ₎ − ΔCt_{(unchallenged} ₐₙᵢₘₐₗ₎
*P < 0.05;
**P < 0.01

### Bioassay 3—Gene Expression Validation

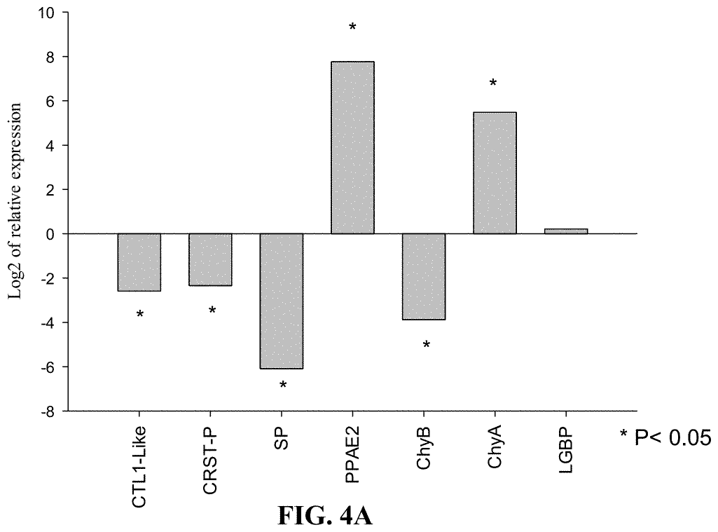

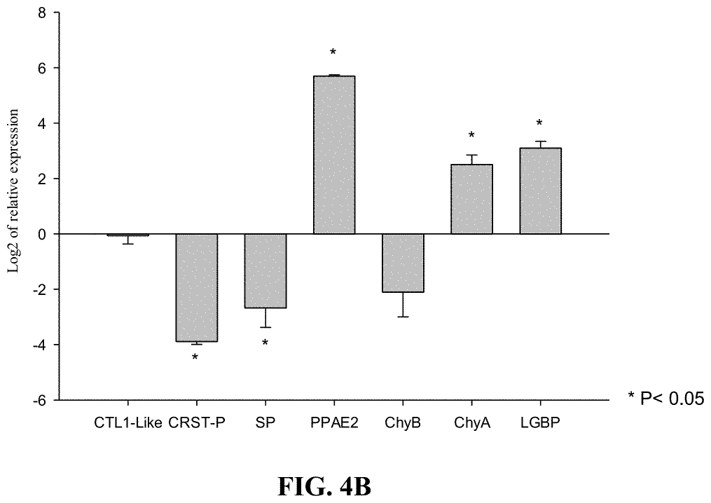

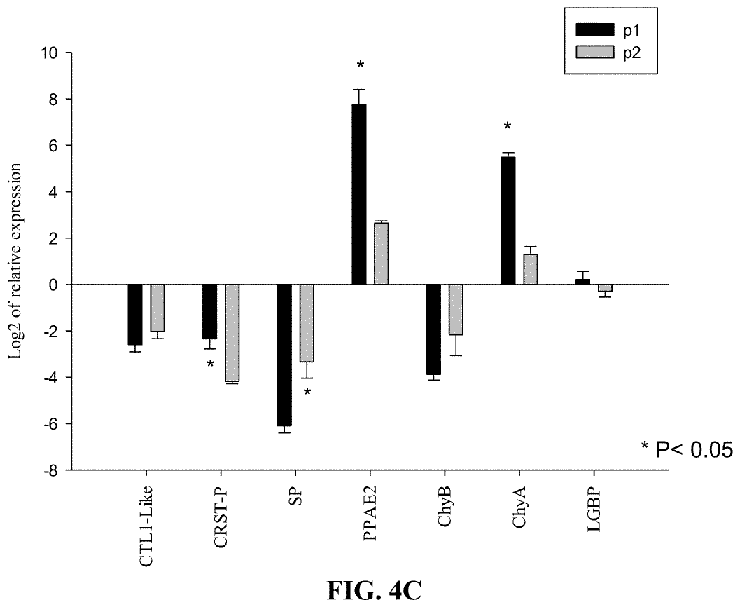

The genes showing significant differences in expression levels between susceptible and tolerant shrimp were selected for validations in Bioassay 3. In the P1 population, the expression of CTL1-like, CRSTP, SP and ChyB were significantly down regulated (P<0.05) meanwhile PPAE2 and ChyA expression levels were significantly up regulated (P<0.05) (FIG. 4A). LGBP expression level was not significant during the experiment (P>0.05) (FIG. 4B).

In P2 population, PPAE2, ChyA and LGBP expression levels were significantly up regulated (P<0.05). In contrast, the expression levels of CRSTP and SP were down regulated (P<0.05) (FIG. 4B). CTL1-like and ChyB expression were not significantly different during the experiment (P>0.05) (FIG. 5B)

By comparison, P1 and P2 challenged animals showed the same expression pattern in both Bioassay 2 and Bioassay 3. The expression levels of CRSTP, PPAE2 and ChyA were significantly higher in P1 population (P<0.05). LGBP expression level was higher in P1 population but not significantly different (P>0.05). Meanwhile, SP had a significant higher expression level in P2 population than P1 population (P<0.05) (FIG. 4C). ChyB expression level was higher in P2 population than P1 population even though there was no significant difference observed during the experiment (P>0.05) (FIG. 4C) (Table 3).

TABLE 3
Differentially expression of 7 immune and metabolic related genes between
susceptible (P1) and resistant/tolerant (P2) population (3^{rd} bio trial)

| | Before | After AHPND | | |
|---|---|---|---|---|
| | challenge- | challenge- | | |
| Genes | P1 (Avg. ΔCt) | P1(Avg. ΔCt) | Log₂2^{-ΔΔCt} | P-value |
| CTL-1-like | 4.18 ± 0.44 | 6.77 ± 0.52 | −2.59 ± 0.32 | 0.006** |
| CRST-P | −0.17 ± 0.43 | 2.17 ± 0.72 | −2.34 ± 0.44 | 0.017* |
| SP | 2.46 ± 0.47 | 8.55 ± 0.51 | −6.09 ± 0.31 | 0.000** |
| PPAE | 12.79 ± 3.73 | 5.03 ± 1.52 | 7.76 ± 0.64 | 0.047* |
| ChyB | −2.95 ± 0.21 | 0.92 ± 0.41 | −3.87 ± 0.25 | 0.000** |
| ChyA | 8.11 ± 0.18 | 2.63 ± 0.34 | 5.48 ± 0.21 | 0.000** |
| LGBP | 10.21 ± 1.95 | 10.00 ± 0.59 | 0.21 ± 0.36 | 0.889 |
| | Before | After AHPND | | |
| | challenge- | challenge- | | |
| Genes | P2 (Avg. ΔCt) | P2 (Avg. ΔCt) | Log₂2^{-ΔΔCt} | P-value |
| CTL-1-like | 6.15 ± 0.52 | 6.21 ± 0.50 | −0.06 | 0.827 |
| CRST-P | 0.12 ± 0.33 | 4.01 ± 0.17 | −3.89 | 0.045* |
| SP | 3.13 ± 0.29 | 5.80 ± 1.15 | −2.67 | 0.033* |
| PPAE | 15.84 ± 2.38 | 10.14 ± 0.02 | 5.70 | 0.027* |
| ChyB | −2.89 ± 0.28 | −0.79 ± 1.46 | −2.10 | 0.117 |
| ChyA | 9.32 ± 0.90 | 6.82 ± 0.56 | 2.50 | 0.029* |
| LGBP | 13.61 ± 0.74 | 10.51 ± 0.40 | 3.10 | 0.007** |

| | Before | After AHPND | After AHPND | | | |
|---|---|---|---|---|---|---|
| | challenge- | challenge- | challenge- | Log₂2^{-ΔΔCt} | Log₂2^{-ΔΔCt} | |
| Genes | P1 (Avg. ΔCt) | P1(Avg. ΔCt) | P2(Avg. ΔCt) | (P1) | (P2) | P-value |
| CTL-1-like | 4.18 ± 0.44 | 6.77 ± 0.52 | 6.21 ± 0.50 | −2.59 ± 0.32 | −2.03 ± 0.31 | 0.332 |
| CRST-P | −0.17 ± 0.43 | 2.17 ± 0.72 | 4.01 ± 0.17 | −2.34 ± 0.44 | −4.18 ± 0.10 | 0.025* |
| SP | 2.46 ± 0.47 | 8.55 ± 0.51 | 5.80 ± 1.15 | −6.09 ± 0.31 | −3.34 ± 0.70 | 0.036* |
| PPAE | 12.79 ± 3.73 | 5.03 ± 1.52 | 10.14 ± 0.02 | 7.76 ± 0.64 | 2.65 ± 0.10 | 0.002** |
| ChyB | −2.95 ± 0.21 | 0.92 ± 0.41 | −0.79 ± 1.46 | −3.87 ± 0.25 | −2.16 ± 0.89 | 0.187 |
| ChyA | 8.11 ± 0.18 | 2.63 ± 0.34 | 6.82 ± 0.56 | 5.48 ± 0.21 | 1.29 ± 0.35 | 0.001** |
| LGBP | 10.21 ± 1.95 | 10.00 ± 0.59 | 10.51 ± 0.40 | 0.21 ± 0.36 | −0.3 ± 0.25 | 0.365 |

ΔCt Was Calculated as Ct_{(target gene)} − Ct_{(EF-1α)}
^{ΔΔCt}Was Calculated as ΔCt_{(challenged animal)} − ΔCt_{(unchallenged animal)}
*P < 0.05;
**P < 0.01

### Discussion

Bacterial pathogenesis in crustaceans is well studied and genes involved in humoral and cellular immunity are known. We decided to take advantage of this background knowledge by measuring the expression of genes that are well known to be involved in defense and metabolic responses during bacterial infections in shrimp. Coincidentally, we had access to AHPND-tolerant lines of P. vannamei and considering the lethal nature of AHPND-causing V. parahaemolyticus we explored if genes known to be involved in other bacterial pathogenesis in shrimp are also involved in AHPND pathogenesis. To our knowledge, a recently published paper is the first report of the development of AHPND resistant/tolerant lines of P. vannamei, Aranguren Caro et al¹⁵, and as of today, there is no report of looking into the gene expression profiles of AHPND-tolerant vs. susceptible lines.

It is now widely accepted that the etiology of AHPND is the insecticidal binary toxin-like genes carried by plasmid DNA in Vibrio spp.^{11,12,26} An AHPND resistant/tolerant shrimp line would be ideal in controlling the disease in shrimp aquaculture. Tinwongger and colleagues¹⁶ showed that shrimp exposed to formalin killed cells (FKC) of AHPND causing V. parahaemolyticus can survive upon AHPND challenge, and anti-lipopolysaccharide factor AV-R isoform (LvALF AV-R) showed significantly higher expression in the hepatopancreas from the survivor. Interestingly, only four out of two hundred shrimp (2%) survived after feeding with FKC diet, and only three shrimp from the survivor group were used for gene expression analysis. Despite examining a limited number of animals, the authors were successful in identifying genes that could be potentially involved in AHPND pathogenesis. In this study, three bioassays were performed. Bioassay 1 was performed to identify an AHPND tolerant line, P2, using mortality and histopathology as end point data of the bioassay. The P2 population suffered 28% mortality compared to the susceptible line P1 that experienced 87.5% mortality (FIG. 5). The bioassay was repeated using the same AHPND-susceptible (P1) and tolerant line (P2) (i.e., Bioassay 2) to examine the mRNA expression of twelve candidate immune and metabolic genes. When the expression of these genes were compared before and after challenge within a population, P1 and P2, a number of genes showed differential expression. However, when the expression profiles were compared between P1 and P2 populations after AHPND-challenge, seven candidate genes showed differential expression. These genes are likely involved in AHPND pathogenesis. In order to further validate the expression of these seven genes, a third bioassay was conducted (i.e. Bioassay 3) and samples were collected for the gene expression validation. The data shed light on the molecular basis of AHPND pathogenesis and enabled to identify potential markers for AHPND tolerance/susceptibility, as discussed below.

### Bioassay 1

The mortality data in the Bioassay 1 showed that the survival rate of the P2 population was three times higher than P1 population indicating that P2 population is indeed an AHPND tolerant line. The mortality data was consistent with the histopathology findings that cellular damage in the hepatopancreas was far greater in animals from the P1 compared to the P2 population. For example, sloughing of the epithelial cells in the hepatopancreatic tubules followed by massive infiltration of bacterial cells that are considered pathognomonic for AHPND was clearly evident by H&E histology in animals from P1 population (FIGS. 1A-1D), whereas in the P2 population, the lesions present in the hepatopancreas resembled more of a chronic infection as seen during SHPN. The differences in mortality and histopathology data led to examining the expression profiles of twelve metabolic and immune-related genes in these two populations.

Gene Expression in P. vannamei from Bioassay 2 vs. Bioassay 3

The expression of twelve candidate genes known to be involved in other bacterial diseases were examined to determine if similar genes are involved in AHPND pathogenesis. It was interesting to note that there was no significant difference in the expression levels of pirA or pirB toxin genes between the P1 and P2 populations suggesting that the animals from the two populations were exposed to equivalent levels of toxin. Thus, the difference in tolerance was most likely due to the difference in immune response between the two populations.

It is known that shrimp, like other invertebrates, elicit cellular and humoral immune responses when exposed to microbes or non-self-protein containing pathogen associated molecular patterns (PAMP)^{27,28}. PAMP is easily recognized by pattern recognition proteins including BGBP, LGBP and CTL^{19,29-32}. Several studies indicate that the expression of BGBP, LGBP and CTL are up-regulated in shrimp challenged with pathogens such as bacteria, viruses and fungi³³⁻³⁶.

In Bioassay 2, when the mRNA expressions of BGBP, LGBP and CTL1-like genes were compared between AHPND-susceptible P1 and AHPND-tolerant P2 populations, LGBP and CTL1-like genes were found to be upregulated in P1 compared to P2 population. However, there was no difference in BGBP expression between the two populations (FIG. 3C). Interestingly, in Bioassay 3, although LGBP expression was higher in P1 than P2 populations as in Bioassay 2 samples, the difference in expression was not statistically significant (p=0.365). In contrary, CTL1-like gene did not show any differential expression between the two populations (FIGS. 4A-4C) as observed in samples derived from Bioassay 2. These discrepancies highlight two important facts: (i) it is critical to validate gene expression data with biological samples derived from independent bioassays, and (ii) it is important to validate initial findings with larger sample sets, as in Bioassay 3 compared to Bioassay 2. These gives further credence to the mRNA expression findings reported in this study.

In shrimp, upon microbial infection pattern recognition protein(s) circulating in the hemolymph triggers immune response by activating proPO cascade and releasing antimicrobial peptides such as PEN and CRSTP (SEQ ID NO: 34) to eliminate the invading pathogen(s)²⁰. Although PEN and CRSTP (SEQ ID NO: 34) were shown to have antimicrobial activities against Vibrio spp. and Gram positive bacteria in penaeid shrimp^{21,23,37} neither of these genes were significantly upregulated in the challenged animals in Bioassay 2. In shrimp, PEN2 is mostly detected in hemocytes and to a lesser level in hepatopancreas. Since we examined the gene expression in hepatopancreas tissue, it is possible that for this reason PEN2 was not found to be differentially expressed. Interestingly, CRSTP (SEQ ID NO: 34) which is also predominantly expressed in hemocytes and less in hepatopancreas showed significantly higher expression in AHPND susceptible P1 compared to AHPND tolerant P2 populations in both Bioassays 2 and 3. It remains to be determined if higher CRSTP (SEQ ID NO: 34) expression in the susceptible population is due to increased bacterial cell deaths and consequently the release of PirAB^{VP} toxin in the infected animals.

The proPO activation is an important event in crustacean immunity to eliminate pathogens from the circulatory system^{38,39} The final event in the proPO activation process is the conversion of proPO to phenoloxidase (PO) by the PPAE^{25,40,41}. In both Bioassays 2 and 3, PPAE2 showed significantly higher expression after AHPND challenge in P1 compared to P2 populations. The finding is consistent with a previously published report in P. monodon where PPAE2 expression in the stomach showed significant upregulation at 24 hour post-challenge with AHPND causing V. parahaemolyticus⁴². In a separate study, Apitanyasai et al.,⁴³ suggested an overreaction of the proPO cascade causes damage to host cells during AHPND infection and leads to higher mortality. The higher expression of PPAE2 in AHPND susceptible P1 population supports this observation. Taken together, this evidence suggests that upon infection with Vp_{AHPND}, not only the expression of antimicrobial peptide like CRSTP (SEQ ID NO: 34) but also the genes involved in the proPO pathway are elevated in AHPND susceptible compared to tolerant animals.

It is known that the activation of proPO leads to production of quinones and other intermediate reactions which polymerizes quinones to melanin resulting in pathogen capsulation⁴⁴. Quinone, however, also causes cell death by inducing reactive oxygen species (ROS) production⁴⁵⁻⁴⁷. In crustacean, ROS can be scavenged by an anti-oxidant system involving enzymes of the SOD family⁴⁸⁻⁵⁰. The EC-SOD expression in the animals studied was not significantly modulated upon AHPND challenge. The proPO cascade is also modulated by a series of protease inhibitors such as KPI and SEP8 to prevent the over activation^{51,52}. Again, both KPI and SEP8 mRNA levels in animals from the P1 and P2 populations were not significantly different. It is tempting to speculate that for the lack of modulation of efforts in EC-SOD, proPO cascade activation led to cell toxicity more in P1 compared to P2 population. It is also possible that killing of Vp_{AHPND} cells leads to further release of PirAB toxin from the inactivated bacterial cells exerting lethal effects in genetically susceptible animals.

The involvement of PO activity in the susceptibility of invertebrates due to bacterial toxins has been well documented in many insect species. For example, cabbage loopers (Trichoplusia ni) is inherently susceptible to the Cry toxin secreted from Bacillus thuringiensis. However, the Cry toxin resistance is negatively correlated with the PO activity in B. thuringiensis challenged T.ni⁵³. In addition, the effectiveness of biological insecticide is also higher in lepidopteran species that show high PO activity⁵⁴. Recently, it has been reported that during VP_{AHPND} infection resulting in AHPND, reduction in activation of the proPO system by a serine protease inhibitor, LvSerpin7, results in reduction of the toxic effects compared to an unregulated activation of the PO cascade⁴³.

Apart from the difference in immune gene expression, the expression of metabolic genes also showed differences between healthy and AHPND-challenged animals in each population and between challenged animals in populations P1 vs P2. The expression of ChyB and SP were down regulated whereas ChyA expression was up regulated in AHPND challenged shrimp in both P1 and P2 populations and in both Bioassays 2 and 3. These results are consistent with the findings from a recent study in which SP was shown to be upregulated in AHPND tolerant P. monodon¹⁸. In addition, Chymotrypsin is upregulated in AHPND susceptible P. vannamei after 24 hour of infection¹⁷ which is also in agreement with our findings.

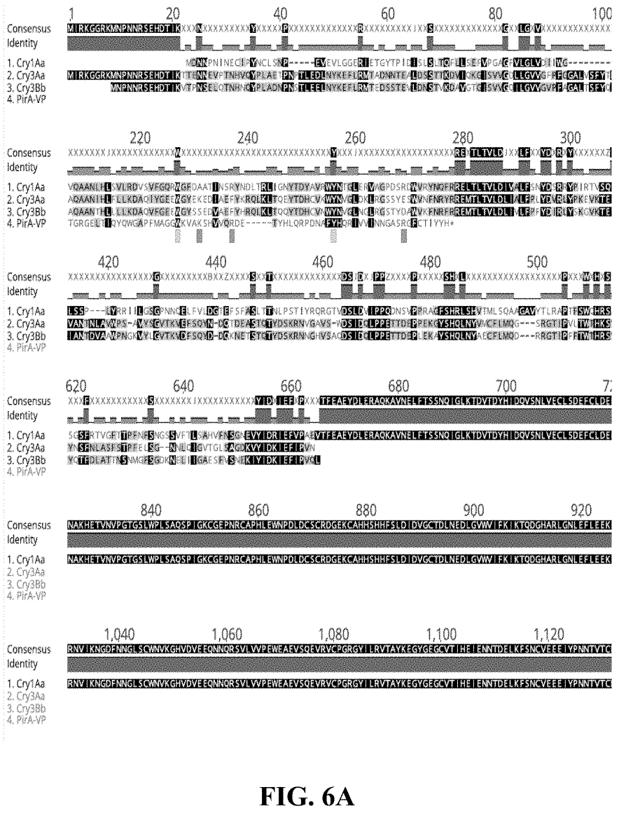

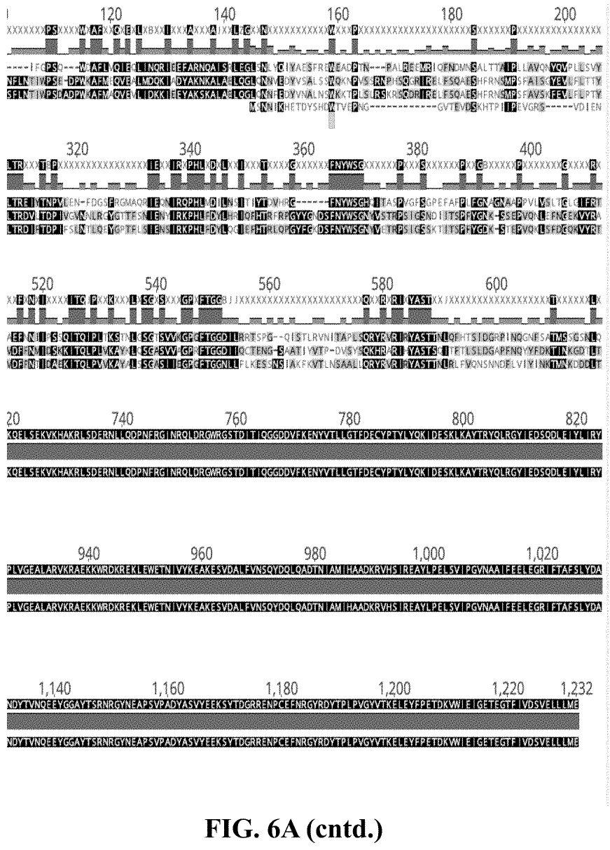

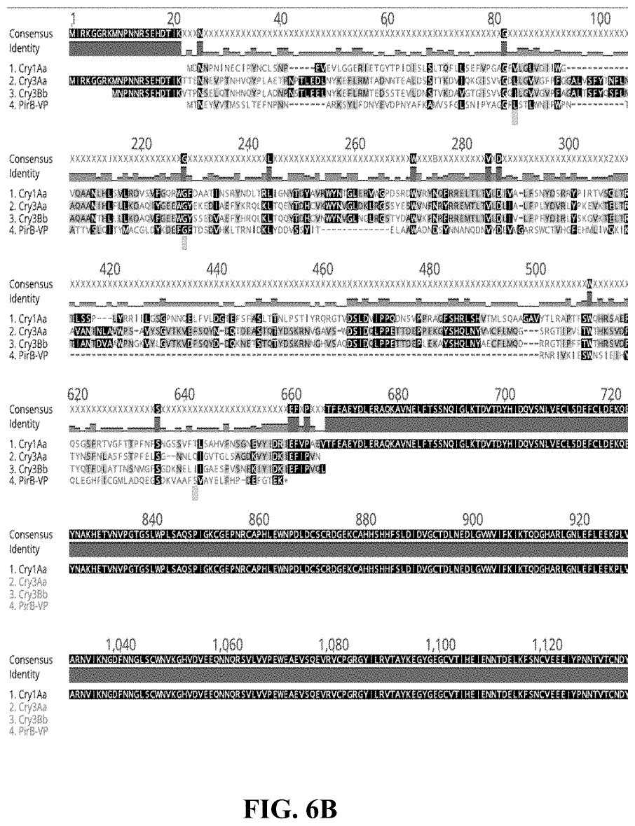

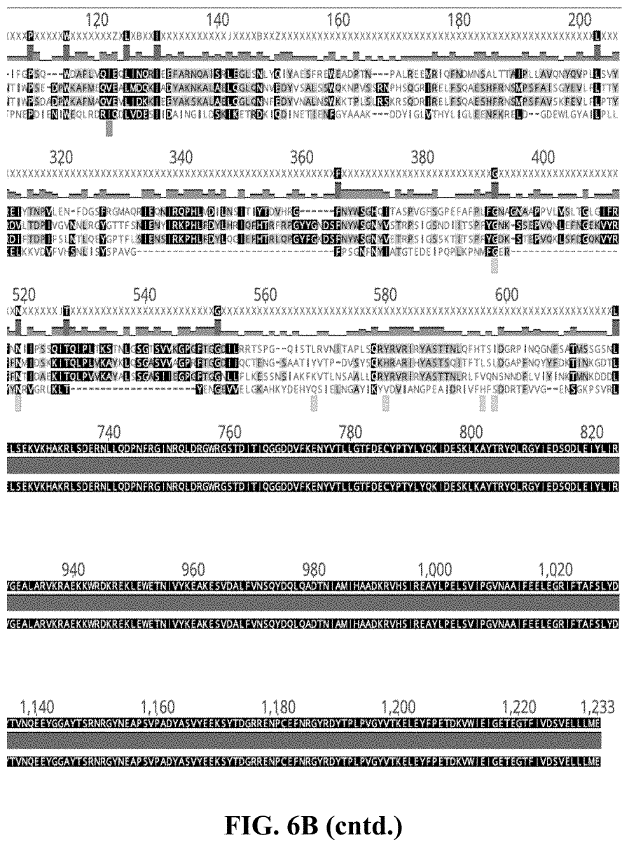

Recently, the crystal structure of PirAB^{VP} toxin shows homology to the structure of the Cry toxin released by B. thuringiensis⁵⁵, and it is known that the Cry protein is activated by protease enzymes⁵⁵⁻⁵⁷. The tertiary structure of PirAB^{VP} involves a heterotetrameric interaction between two PirA^{VP} and two PirB^{VP}. It has been hypothesized that PirA^{VP} plays a role in receptor binding while PirB^{VP} is involved in pore formation in the cell membrane⁵⁸. In silico analysis (https://web.expasy.org/peptide_cutter/) showed that the PirA^{VP} toxin contains twelve and nine putative sites that are likely to be cleaved by chymotrypsin and trypsin, respectively (FIGS. 6A-6B). Meanwhile, PirB^{VP} toxin contains 53 and 36 sites cleaved by chymotrypsin and trypsin, respectively. Multiple alignment between Cry toxin and PirA^{VP} showed that 3 out of 12 and 3 out of 9 cleaved sites for chymotrypsin and trypsin were conserved. Meanwhile, the multiple alignment between Cry toxin and PirB^{VP} showed that 9 out of 53 and 1 out of 36 cleaved sites for chymotrypsin and trypsin were conserved (FIGS. 6A-6B). It is interesting to note that SP expression showed higher levels in the AHPND tolerant than AHPND susceptible P. vannamei in this study and this enzyme belongs to trypsin family⁵⁹. It remains to be determined if SP is involved in cleaving PirAB^{VP} toxins to de-activate the toxin. If so, a higher expression of this enzyme in AHPND tolerant population (P2) may prevent the activation of toxin from exerting a lethal effect.

To summarize, we compared the gene expression profiles of two populations of P. vannamei that differ in susceptibility to AHPND. The two populations showed a major difference in survival upon experimental challenge. The difference in susceptibility was further evidenced by the differences observed by histopathology. In order to understand the molecular mechanisms governing tolerance and susceptibility, a set of candidate genes that are known to be involved in bacterial pathogenesis and in metabolism in crustaceans were evaluated. Seven genes that showed differential expression in Bioassay 2 were further evaluated in a follow-up challenge, Bioassay 3. The pattern of differential expression between the susceptible (P1) and tolerant (P2) population in Bioassays 2 and 3 were in agreement. Despite the fact that the mRNA expression of only handful genes were measured and a limited number of animals were screened, the information, albeit limited, provides valuable insight in V. parahaemolyticus pathogenesis and sheds light on how susceptible and tolerant populations of P. vannamei respond differently to Vp_{AHPND}. To our knowledge, this is the first report looking into the differences in gene expression profiles between AHPND tolerant and susceptible lines in P. vannamei.

### REFERENCES RELEVANT TO THE DESCRIPTION AND EXAMPLES

1. Peña, L. D. De et al. Acute hepatopancreatic necrosis disease (AHPND) outbreaks in Penaeus vannamei and P. monodon cultured in the Philippines. Dis. Aquat. Org. 116, 251-254 (2015).
2. Dhar, A. K. et al. First report of acute hepatopancreatic necrosis disease (AHPND) occurring in the USA. Dis. Aquat. Org. 132, 241-247 (2019).
3. Eun, J. et al. Genomic and histopathological characteristics of Vibrio parahaemolyticus isolated from an acute hepatopancreatic necrosis disease outbreak in Paci fi c white shrimp (Penaeus vannamei) cultured in Korea. Aquaculture 524, 735284 (2020).
4. Joshi, J. et al. Variation in Vibrio parahaemolyticus isolates from a single Thai shrimp farm experiencing an outbreak of acute hepatopancreatic necrosis disease (AHPND).
Aquaculture 428-429, 297-302 (2014).
5. Nunan, L., Lightner, D., Pantoja, C. & Gomez-jimenez, S. Detection of acute hepatopancreatic necrosis disease (AHPND) in Mexico. Dis. Aquat. Organ. 111, 81-86 (2014).
6. Thitamadee, S. et al. Review of current disease threats for cultivated penaeid shrimp in Asia. Aquaculture 452, 69-87 (2016).
7. M M E, E., M M, A., N J, P., M K, B. & Rahman, M. Molecular Identification of AHPND Positive Vibrio Parahaemolyticus causing an outbreak in South-West Shrimp Farming Regions of Bangladesh. J. Bangladesh Acad Sci 41, 127-135 (2017).
8. Han, J. E., Tang, K. F. J., Aranguren, L. F. & Piamsomboon, P. Characterization and pathogenicity of acute hepatopancreatic necrosis disease natural mutants, pirABvp(−) V. parahaemolyticus, and pirABvp(+) V. campbellii strains. Aquaculture 470, 84-90 (2017).
9. Tran, L. et al. Determination of the infectious nature of the agent of acute hepatopancreatic necrosis syndrome affecting penaeid shrimp. Dis. Aquat. Organ. 105, 45-55 (2013).
10. Liu, L. et al. Investigation of Vibrio alginolyticus, V. harveyi, and V. parahaemolyticus in large yellow croaker, Pseudosciaena crocea (Richardson) reared in Xiangshan Bay, China. Aquac. Reports 3, 220-224 (2016).
11. Jesus, M. De et al. pir A- and pirB-like gene identification in Micrococcus luteus strains in Mexico. J. Fish Dis. 1667-1673 (2018). doi:10.1111/jfd.12874
12. Han, J. E., Tang, K. F. J., Tran, L. H. & Lightner, D. V. Photorhabdus insect-related (Pir) toxin-like genes in a plasmid of Vibrio parahaemolyticus, the causative agent of acute hepatopancreatic necrosis disease (AHPND) of shrimp. Dis. Asian Aquac. 113, 33-40 (2015).
13. Soto-rodriguez, S. A., Gomez-gil, B., Lozano-olvera, R., Betancourt-lozano, M. & Morales-covarrubias, M. S. Field and Experimental Evidence of Vibrio parahaemolyticus as the Causative Agent of Acute Hepatopancreatic Necrosis Disease of Cultured Shrimp (Litopenaeus vannamei) in Northwestern Mexico. Appl. Environ. Microbiol. 81, 1689-1699 (2015).
14. Lai, H. et al. Fish & Shell fish Immunology Pathogenesis of acute hepatopancreatic necrosis disease (AHPND) in shrimp. Fish Shellfish Immunol. 47, 1006-1014 (2015).
15. Aranguren Caro, L. F., Mai, H. N., Noble, B. & Dhar, A. K. Acute hepatopancreatic necrosis disease (VP AHPND), a chronic disease in shrimp (Penaeus vannamei) population raised in Latin America. J. Invertebr. Pathol. 174, 107424 (2020).
16. Tinwongger, S., Thawonsuwan, J., Kondo, H. & Hirono, I. Fish and Shell fish Immunology Identification of an anti-lipopolysaccharide factor AV-R isoform (Lv ALF AV-R) related to Vp_PirAB-like toxin resistance in Litopenaeus vannamei. Fish Shellfish Immunol. 84, 178-188 (2019).
17. Vela, E. et al. Transcriptomic analysis of Pacific white shrimp (Litopenaeus vannamei, Boone 1931) in response to acute hepatopancreatic necrosis disease caused by Vibrio parahaemolyticus. PLoS One e0220993, 1-28 (2019).
18. Soo Christie, Chiew, T., Devadas, S., Shariff, M., Din, M. & Bhassu, S. Differential transcriptome analysis of the disease tolerant Madagascar-Malaysia crossbred black tiger shrimp, Penaeus monodon hepatopancreas in response to acute hepatopancreatic necrosis disease (AHPND) infection: inference on immune gene response. Gut Pathog. 11, 1-13 (2019).
19. Lee, S. Y., Wang, R. & So, K. A Lipopolysaccharide- and ^{N}_{L}-1,3-Glucan-binding Protein from Hemocytes of the Freshwater Crayfish Pacifastacus leniusculus. J. Biol. Chem. 275, 1337-1343 (2000).
20. Bernard, D. & Söderhäll, K. β-1,3-Glucan-Binding Proteins from Plasma of the Fresh-Water Crayfishes Astacus astacus and Procambarus clarkii Author (s): Bernard Duvic and Kenneth Söderhäll Published by: Oxford University Press on behalf of The Crustacean Society Stable URL: htt. J. Crustac. Biol. 13, 403-408 (1993).
21. Shockey, J. E. et al. The role of crustins in Litopenaeus vannamei in response to infection with shrimp pathogens: An in vivo approach. Dev. Comp. Immunol. 33, 668-673 (2009).
22. Amparyup, P., Charoensapsri, W. & Tassanakajon, A. Fish & Shell fish Immunology Prophenoloxidase system and its role in shrimp immune responses against major pathogens. Fish Shellfish Immunol. 34, 990-1001 (2013).
23. Vandenbulcke, F., Saulnier, D., Bache, E. & Mun, M. Expression and distribution of penaeidin antimicrobial peptides are regulated by haemocyte reactions in microbial challenged shrimp. Eur. J. Biochem. 2689, 2678-2689 (2002).
24. Charoensapsri, W., Amparyup, P., Hirono, I., Aoki, T. & Tassanakajon, A. Pm PPAE2, anew class of crustacean prophenoloxidase (proPO)-activating enzyme and its role in PO activation. Dev. Comp. Immunol. 35, 115-124 (2011).
25. Livak, K. J. & Schmittgen, T. D. Analysis of Relative Gene Expression Data Using Real-Time Quantitative PCR and the 2(−Delta Delta C(T)) Method. METHODS 408, 402-408 (2001).
26. Kondo, H., Van, P. T. & Dang, L. T. Draft Genome Sequence of Non-Vibrio parahaemolyticus Acute Diseased Shrimp in Vietnam. Genome Announc. 3, 2014-2015 (2015).
27. Hauton, C. The scope of the crustacean immune system for disease control. J. Invertebr. Pathol. 110, 251-260 (2012).
28. Jiravanichpaisal, P., Luel, B. & So, K. Cell-mediated immunity in arthropods: Hematopoiesis, coagulation, melanization and opsonization. Immunobiology 211, 213-236 (2006).
29. Wang, X. & Wang, J. Fish & Shell fish Immunology Pattern recognition receptors acting in innate immune system of shrimp against pathogen infections. Fish Shellfish Immunol. 34, 981-989 (2013).
30. Yu, X. & Kanost, M. R. Immulectin-2, a pattern recognition receptor that stimulates hemocyte encapsulation and melanization in the tobacco hornworm, Manduca sexta. Dev. Comp. Immunol. 28, 891-900 (2004).
31. Beschin, A. et al. Identification and Cloning of a Glucan- and Lipopolysaccharide-binding Protein from Eisenia foetida Earthworm Involved in the Activation of Prophenoloxidase Cascade *. J. Biol. Chem. 273, 24948-24954 (1998).
32. Kim, Y. et al. Gram-negative Bacteria-binding Protein, a Pattern Recognition Receptor for Lipopolysaccharide and B-1,3-Glucan That Mediates the Signaling for the Induction of Innate Immune Genes in Drosophila melanogaster Cells *. J. Biol. Chem. 275, 32721-32727 (2000).
33. Roux, M. M., Pain, A., Klimpel, K. R. & Dhar, A. K. The Lipopolysaccharide and ^{N}_{L}B-1,3-Glucan Binding Protein Gene Is Upregulated in White Spot Virus-Infected Shrimp (Penaeus stylirostris). J. Virol. 76, 7140-7149 (2002).
34. Zhao, Z. et al. A Novel C-Type Lectin from the Shrimp Litopenaeus vannamei Possesses Anti-White Spot Syndrome Virus Activity. J. Virol. 83, 347-356 (2009).
35. Cheng, W., Liu, C., Tsai, C. & Chen, J. Molecular cloning and characterisation of a pattern recognition molecule, lipopolysaccharide- and b-1,3-glucan binding protein (LGBP ) from the white shrimp Litopenaeus vannamei. Fish Shellfish Immunol. 18, 297-310 (2005).
36. Amparyup, P., Sutthangkul, J., Charoensapsri, W. & Tassanakajon, A. Pattern Recognition Protein Binds to Lipopolysaccharide and B-1,3-Glucan and Activates Shrimp Prophenoloxidase. J. Biol. Chem. 287, 10060-10069 (2012).
37. Destoumieux, D., Bulet, P., Strub, J., Dorsselaer, A. Van & Bache, E.
Recombinant expression and range of activity of penaeidins, antimicrobial peptides from penaeid shrimp. enviromental Microbiol. 346, 335-346 (1999).
38. Sritunyalucksana, K. & Söderhäll, K. The proPO and clotting system in crustaceans. Aquaculture 191, 53-69 (2000).
39. Cerenius, L., Kawabata, S., Lee, B. L. & Nonaka, M. Proteolytic cascades and their involvement in invertebrate immunity. Trends Biochem. Sci. 35, 575-583 (2010).
40. Wang, R., Lee, S. Y., Cerenius, L. & So, K. Properties of the prophenoloxidase activating enzyme of the freshwater crayfish, Pacifastacus leniusculus. Eur. J. Biochem. 902, 895-902 (2001).
41. Charoensapsri, W., Amparyup, P., Hirono, I., Aoki, T. & Tassanakajon, A.
Gene silencing of a prophenoloxidase activating enzyme in the shrimp, Penaeus monodon, increases susceptibility to Vibrio harveyi infection. Dev. Comp. Immunol. 33, 811-820 (2009).
42. Soonthornchai, W., Chaiyapechara, S. & Klinbunga, S. Differentially expressed transcripts in stomach of Penaeus monodon in response to AHPND infection. Dev. Comp. Immunol. 65, 53-63 (2016).
43. Apitanyasai, K., Chang, C., Hann, T., Siong, Y. & Liou, J. Penaeus vannamei serine proteinase inhibitor 7 (Lv Serpin7) acts as an immune brake by regulating the proPO system in AHPND-affected shrimp. Dev. Comp. Immunol. 106, 103600 (2020).
44. Cerenius, L., Lee, B. L. & So, K. The proPO-system: pros and cons for its role in invertebrate immunity. Trends Immunol. 263-271 (2008). doi:10.1016/j.it.2008.02.009
45. Hergenhahn, H., Aspant, A. & Söderhälltt, K. Purification and characterization of a high-MA proteinase inhibitor of pro-phenol oxidase activation from crayfish plasma. Biochem. J. 248, 223-228 (1987).
46. Kan, H. et al. Molecular Control of Phenoloxidase-induced Melanin Synthesis in an Insect * □. J Biol. Chem. 283, 25316-25323 (2008).
47. Saibu, M., Sagar, S., Green, I., Ameer, F. & Meyer, M. Evaluating the Cytotoxic Effects of Novel Quinone Compounds. Anticancer Res. 4086, 4077-4086 (2014).
48. Fridovich, I. SUPEROXIDE RADICAL AND SUPEROXIDE DISMUTASES. Annu. Rev. Biochem. 97-112 (1995). doi:10.1146/annurev.bi.64.070195.000525
49. Hart, P. J. et al. A Structure-Based Mechanism for Copper-Zinc Superoxide Dismutase †‡. Biochemistry 2167-2178 (1999). doi:10.1021/bi982284u
50. Chakravarthy, N., Aravindan, K. & Kalaimani, N. Intracellular Copper Zinc Superoxide dismutase (icCuZnSOD) from Asian seabass (Lates calcarifer): Molecular cloning, characterization and gene expression with reference to Vibrio anguillarum infection. Dev. Comp. Immunol. 36, 751-755 (2012).
51. Wedde, M., Weise, C., Kopacek, P., Franke, P. & Vilcinskas, A. Purification and characterization of an inducible metalloprotease inhibitor from the hemolymph of greater wax moth larvae, Galleria mellonella. Eur. J Biochem. 225, 535-543 (1998). 52. Kanost, M. R. Serine proteinase inhibitors in arthropod immunity. Dev. Comp. Immunol. 23, 291-301 (1999).
53. Ericsson, J. D., Janmaat, A. F., Lowenberger, C. & Myers, J. H. Is decreased generalized immunity a cost of Bt resistance in cabbage loopers Trichoplusia ni ? J. Invertebr. Pathol. 100, 61-67 (2009).
54. Damas, G. & Oppert, B. Comparative evaluation of phenoloxidase activity in different larval stages of four lepidopteran pests after exposure to Bacillus thuringiensis. J. Insect Sci. 12, 1-11 (2012).
55. Lin, S., Hsu, K. & Wang, H. Structural Insights into the Cytotoxic Mechanism of Vibrio parahaemolyticus PirA vp and PirB vp Toxins. Mar. Drugs 15, 9-12 (2017).
56. Loseva, O. et al. Changes in protease activity and Cry3Aa toxin binding in the Colorado potato beetle: implications for insect resistance to Bacillus thuringiensis toxins. Insect Biochem. Mol. Biol. 32, 567-577 (2002).
57. Sa, J., Real, M. D. & Bravo, A. Role of toxin activation on binding and pore formation activity of the Bacillus thuringiensis Cry3 toxins in membranes of Leptinotarsa decemlineata (Say). Biochim. Biophys. Acta 1660, 99-105 (2004).
58. Lin S J et al. Structural Insights to the Heterotetrameric Interaction between the Vibrio parahaemolyticus PirA vp and PirB vp Toxins and Activation of the Cry-Like Pore-Forming Domain. Toxins (Basel). 11, (2019).
59. Vargas-albores, F., So, K. & Jime, F. Characterisation of a serine proteinase from Penaeus vannamei haemocytes. Fish Shellfish Immunol. 18, 101-108 (2005).
60. Cheng W, Liu C H, Tsai C H, Chen J C. Molecular cloning and characterisation of a pattern recognition molecule, lipopolysaccharide- and β-1,3-glucan binding protein (LGBP) from the white shrimp Litopenaeus vannamei. Fish Shellfish Immunol (2005) 18:297-310. doi:10.1016/j.fsi.2004.08.002
61. Lin Y C, Vaseeharan B, Chen J C. Identification and phylogenetic analysis on lipopolysaccharide and β-1,3-glucan binding protein (LGBP) of kuruma shrimp Marsupenaeus japonicus. Dev Comp Immunol (2008) 32:1260-1269. doi:10.1016/j.dci.2008.05.003
62. Qi C, Wang L, Liu M, Jiang K, Wang M, Zhao W, Wang B.
Transcriptomic and morphological analyses of Litopenaeus vannamei intestinal barrier in response to Vibrio parahaemolyticus infection reveals immune response signatures and structural disruption. Fish Shellfish Immunol (2017) 70:437-450. doi:10.1016/j.fsi.2017.09.004
63. Roux M M, Pain A, Klimpel K R, Dhar A K. The Lipopolysaccharide and β-1,3-Glucan Binding Protein Gene Is Upregulated in White Spot Virus-Infected Shrimp (Penaeus stylirostris). J Virol (2002) 76:7140-7149. doi:10.1128/jvi.76.14.7140-7149.2002
64. Vargas-Albores F, Jiménez-Vega F, Söderhäll K. A plasma protein isolated from brown shrimp (Penaeus califoriensis) which enhances the activation of prophenoloxidase system by β-1,3-glucan. Dev Comp Immunol (1996) 20:299-306. doi:10.1016/S0145-305X(96)00007-9
65. Cerenius L, Liang Z, Duvic B, Keyser P, Hellman U, Palva E T, Iwanaga S, Söderhäll K. Structure and biological activity of a 1,3-β-D-glucan-binding protein in crustacean blood. J Biol Chem (1994) 269:29462-29467. doi:10.1016/s0021-9258(18)43902-6
66. Zhao Z Y, Yin Z X, Xu X P, Weng S P, Rao X Y, Dai Z X, Luo Y W, Yang G, Li Z S, Guan H J, et al. A Novel C-Type Lectin from the Shrimp Litopenaeus vannamei Possesses Anti-White Spot Syndrome Virus Activity. J Virol (2009) 83:347-356. doi:10.1128/jvi.00707-08
67. Liu Y C, Li F H, Dong B, Wang B, Luan W, Zhang X J, Zhang L S, Xiang J H. Molecular cloning, characterization and expression analysis of a putative C-type lectin (Fclectin) gene in Chinese shrimp Fenneropenaeus chinensis. Mol Immunol (2007) 44:598-607. doi:10.1016/j.molimm.2006.01.015
68. Zheng J, Mao Y, Su Y, Wang J. Identification of two novel C-type lectins involved in immune defense against white spot syndrome virus and Vibrio parahaemolyticus from Marsupenaeus japonicus. Aquaculture (2020) 519:734797. doi:10.1016/j.aquaculture.2019.734797
69. Li M, Ma C, Zhu P, Yang Y, Lei A, Chen X, Liang W, Chen M, Xiong J, Li C. A new crustin is involved in the innate immune response of shrimp Litopenaeus vannamei. Fish Shellfish Immunol (2019) 94:398-406. doi:10.1016/j.fsi.2019.09.028
70. Li S, Lv X, Yu Y, Zhang X, Li F. Molecular and Functional Diversity of Crustin-Like Genes in the Shrimp Litopenaeus vannamei. Mar Drugs (2020) 18:361. doi:10.3390/md18070361
71. Visetnan S, Supungul P, Tassanakajon A, Donpudsa S, Rimphanitchayakit V. A single WAP domain-containing protein from Litopenaeus vannamei possesses antiproteinase activity against subtilisin and antimicrobial activity against AHPND-inducing Vibrio parahaemolyticus. Fish Shellfish Immunol (2017) 68:341-348. doi:10.1016/j.fsi.2017.07.046
72. Ren Q, Zhao X F, Wang J X. Identification of three different types of serine proteases (one SP and two SPHs) in Chinese white shrimp. Fish Shellfish Immunol (2011) 30:456-466. doi:10.1016/j.fsi.2010.11.009
73. Yang H, Ji T, Xiong H, Zhang Y, Wei W. A trypsin-like serine protease domain of masquerade gene in crayfish Procambarus clarkii could activate prophenoloxidase and inhibit bacterial growth. Dev Comp Immunol (2021) 117:103980. doi:10.1016/j.dci.2020.103980
74. Qiao G, Xu D H, Wang Z, Jang I K, Qi Z, Zhang M, Kim S K. Comparison of immune response of Pacific white shrimp, Litopenaeus vannamei, after multiple and single infections with WSSV and Vibrio anguillarum. Fish Shellfish Immunol (2015) 44:257. doi:10.1016/j.fsi.2015.02.009
75. Zhao Y R, Xu Y H, Jiang H S, Xu S, Zhao X F, Wang J X. Antibacterial activity of serine protease inhibitor 1 from kuruma shrimp Marsupenaeus japonicus. Dev Comp Immunol (2014) 44:261-269. doi:10.1016/j.dci.2014.01.002
76. Liu Y, Hou F, He S, Qian Z, Wang X, Mao A, Sun C, Liu X. Identification, characterization and functional analysis of a serine protease inhibitor (Lvserpin) from the Pacific white shrimp, Litopenaeus vannamei. Dev Comp Immunol (2014) 43:35-46. doi:10.1016/j.dci.2013.10.012
77. Liu Y, Liu T, Hou F, Wang X, Liu X. Lvserpin3 is involved in shrimp innate immunity via the inhibition of bacterial proteases and proteases involved in prophenoloxidase system. Fish Shellfish Immunol (2016) 48:128-135. doi:10.1016/j.fsi.2015.09.039
78. Jang I K, Pang Z, Yu J, Kim S K, Seo H C, Cho Y R. Selectively enhanced expression of prophenoloxidase activating enzyme 1 (PPAE1) at a bacteria clearance site in the white shrimp, Litopenaeus vannamei. BMC Immunol (2011) 12:70. doi:10.1186/1471-2172-12-70
79. Gong J, Xie Y, Yu K, Yang Y, Huang H, Ye H. Characterization and expression analysis of chymotrypsin after bacterial challenge in the mud crab, Scylla paramamosain. Genet Mol Biol (2014) 37:381-388. doi:10.1590/51415-47572014005000007
80. Li X C, Zhang R R, Sun R R, Lan J F, Zhao X F, Wang J X. Three Kazal-type serine proteinase inhibitors from the red swamp crayfish Procambarus clarkii and the characterization, function analysis of hcPcSPI2. Fish Shellfish Immunol (2010) 28:942-951. doi:10.1016/j.fsi.2010.02.011
81. Boonrawd S, Supungul P, Tassanakajon A, Rimphanitchayakit V.
Antimicrobial activity of a serine proteinase inhibitor SPIPm5 from the black tiger shrimp Penaeus monodon. (2018) doi:10.1016/j.fsi.2018.03.044
82. Tian J, Chen J, Jiang D, Liao S, Wang A. Transcriptional regulation of extracellular copper zinc superoxide dismutase from white shrimp Litopenaeus vannamei following Vibrio alginolyticus and WSSV infection. Fish Shellfish Immunol (2011) 30:234-240. doi:10.1016/j.fsi.2010.10.013
83. Hung M N, Shiomi R, Nozaki R, Kondo H, Hirono I. Identification of novel copper/zinc superoxide dismutase (Cu/ZnSOD) genes in kuruma shrimp Marsupenaeus japonicus. Fish Shellfish Immunol (2014) 40:472-477. doi:10.1016/j.fsi.2014.07.030
84. Wu B, Zhang C, Qin X, Shi L, Zhao M. Identification and function of penaeidin 3 and penaeidin 5 in Fenneropenaeus merguiensis. Fish Shellfish Immunol (2019) 89:623-631. doi:10.1016/j.fsi.2019.04.032
85. Vaseeharan B, Shanthi S, Chen J C, Eira E. Molecular cloning, sequence analysis and expression of Fein-Penaeidin from the haemocytes of Indian white shrimp Fenneropenaeus indicus. Results Immunol (2013) 2:35-43. doi:10.1016/j.rinim.2012.02.001

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and can be applied to the essential features herein before set forth.

What is claimed is:

1. A method of detecting an acute hepatopancreatic necrosis disease (AHPND) and/or AHPND susceptible organisms and/or cells therefrom comprising:
detecting in one or more cells from an organism an AHPND signature, wherein the AHPND signature comprises:
(a) one or more genes selected from the group consisting of: SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of: PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof;
(e) one or more genes selected from the group consisting of: CLT1-like, SP, CHYB, CRST-P, PPEA2, and CHYA;
(e) LGBP;
(f) one or more genes selected from the group consisting of: CRST-P, SP, PPEA2, CHYA, and LGBP;
(g) one or more genes selected from the group consisting of: SP, CRST-P, PPAE2, CHYA; or
(h) combinations thereof;
wherein detection of the AHPND signature indicates that the organism or cell(s) thereof is tolerant to AHPND or that the sample contains at least one cell that is tolerant to AHPND or detection of the AHPND signature indicates that the organism or cell(s) thereof is susceptible to AHPND or that the sample contains at least one cell that is susceptible to AHPND.

2. The method of any one of claim 1, wherein the organism is a crustacean.

3. The method of any one of claims 1-2, wherein the organism is a crab, lobster, crayfish, shrimp, prawn, or krill.

4. The method of any of the preceding claims, wherein the sample is obtained from a cell, an organ, a tissue, a bodily fluid, or a combination thereof.

5. The method of any of the preceding claims, wherein the sample is obtained from a hepatopancreas of the organism.

6. The method of any of the preceding claims, wherein the AHPND signature comprises:
(a) decreased expression of SP as compared to a suitable control and increased expression of CHYA, LGBP, and PPEA2 as compared to a suitable control;
(b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal;
(c) increased expression of PPAE2 and CHYA as compared to a suitable control and decreased expression of CTL1-like, CRST-P, SP, and CHYB as compared to a suitable control;
(d) increased expression of CRST-P, PPAE2, and CHYA as compared to a control sample obtained from an AHPND tolerant animal;
(e) decreased expression of LGBP as compared to a suitable control;
(f) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal;
(g) increased expression of PPEA2, CHYA, and LGBP as compared to a suitable control and decreased expression of CRST-P and SP as compared to a suitable control;
(h) increased expression of SP as compared to a control sample obtained from an AHPND susceptible animal and decreased expression of CRST-P, PPEA2, and CHYA as compared to a control sample obtained from an AHPND susceptible animal; or
(i) a combination thereof.

7. The method of claim 6, wherein the AHPND signature comprising (a), (b), (c), (d), or a combination thereof indicates that the organism or cell(s) thereof is susceptible to AHPND or that the sample contains at least one cell that is susceptible to AHPND.

8. The method of any of claims 6-7, wherein the AHPND signature comprising (e), (f), (g), (h), or a combination thereof indicates that the organism or cell(s) thereof is tolerant to AHPND or that the sample contains at least one cell that is tolerant to AHPND.

9. A method of treating or preventing acute hepatopancreatic necrosis disease (AHPND) in an organism or population thereof comprising:
detecting an AHPND tolerant organism and/or AHPND susceptible organism as in any one of claims 1-8 and
(a) administering an effective amount of an agent effective to treat or prevent AHPND infection;
(b) administering an effective amount of an agent effective to increase the tolerance of the organism to AHPND and/or reduce the susceptibility of the organism to AHPND;
(c) perform techniques capable of reducing AHPND in the environment;
(d) perform techniques capable of decreasing the mortality of AHPND in the organism(s);
(e) perform techniques capable of reducing the spread of and/or introduction of AHPND into the organism or population thereof; or
(f) any combination thereof.

10. A method of screening for an agent effective to modify the acute hepatopancreatic necrosis disease (AHPND) tolerance of an organism and/or modify the AHPND susceptibility of an organism, comprising:
contacting an AHPND tolerant cell or an AHPND susceptible cell having an initial cell signature and/or cell state or type with a test agent; and
determining a change in the initial cell signature, a shift in initial cell state or type, or both, wherein a change in the initial cell signature, a shift in initial cell state or type or both identifies, the test agent as an agent effective to modify the AHPND tolerance or susceptibility of the cell, organism, or both, and wherein determining a change in the initial cell signature, a shift in initial cell state or type, or both comprises a method as in any one of claims 1-8.

11. The method of claim 10, wherein the agent is effective to increase the AHPND tolerance, reduce AHPND of a cell, an organism, or both.

12. The method of claim 10, wherein the agent is effective to decrease the AHPND susceptibility of a cell, an organism, or both.

13. A cell, cell population, or progeny thereof comprising:
an acute hepatopancreatic necrosis disease (AHPND) signature, wherein the AHPND signature comprises:
(a) one or more genes selected from the group consisting of SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof;
(e) one or more genes selected from the group consisting of: CLT1-like, SP, CHYB, CRST-P, PPEA2, and CHYA;
(e) LGBP;
(f) one or more genes selected from the group consisting of CRST-P, SP, PPEA2, CHYA, and LGBP;
(g) one or more genes selected from the group consisting of: SP, CRST-P, PPAE2, CHYA; or
(h) a combination thereof.

14. The cell population, or progeny thereof of claim 13, wherein the cell is a crustacean cell.

15. The cell population, or progeny thereof of claim 14, wherein the crustacean is a crab, lobster, crayfish, shrimp, prawn, or krill.

16. The cell population, or progeny thereof of any one of claims 13-15, wherein the AHPND signature comprises:
(a) decreased expression of SP as compared to a suitable control and increased expression of CHYA, LGBP, and PPEA2 as compared to a suitable control;
(b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal;
(c) increased expression of PPAE2 and CHYA as compared to a suitable control and decreased expression of CTL1-like, CRST-P, SP, and CHYB as compared to a suitable control;
(d) increased expression of CRST-P, PPAE2, and CHYA as compared to a control sample obtained from an AHPND tolerant animal;
(e) decreased expression of LGBP as compared to a suitable control;
(f) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal;
(g) increased expression of PPEA2, CHYA, and LGBP as compared to a suitable control and decreased expression of CRST-P and SP as compared to a suitable control;
(h) increased expression of SP as compared to a control sample obtained from an AHPND susceptible animal and decreased expression of CRST-P, PPEA2, and CHYA as compared to a control sample obtained from an AHPND susceptible animal; or
(i) a combination thereof.

17. The cell population, or progeny thereof of claim 16, wherein the AHPND signature comprising (a), (b), (c), (d), or a combination thereof indicates that the organism or cell(s) thereof is susceptible to AHPND or that the sample contains at least one cell that is susceptible to AHPND.

18. The cell population, or progeny thereof of any one of claims 16-17, wherein the AHPND signature comprising (e), (f), (g), (h), or a combination thereof indicates that the organism or cell(s) thereof is tolerant to AHPND or that the sample contains at least one cell that is tolerant to AHPND.

19. A modified non-human organism comprising:
one or more modified genes or expression thereof, wherein the one or more genes are one or more genes of an AHPND signature and are
(a) one or more genes selected from the group consisting of: SEP8, BGBP, CRST P, CTL1-like, KPI, LGBP, EC-SOD, SP, PEN2, PPAE2, CHYA; CHYB, PirA-like, PriB-like, EF1-aplha, and any combination thereof;
(b) one or more genes selected from the group consisting of: SEP8, BGBP, CRST-P CTL1-like, KPI, LGBP, EC-SOD, SP, PEN2, PPAE2, CHYA, CHYB, and any combination thereof;
(c) one or more genes selected from the group consisting of: PPAE2, LGBP, CHYA, SP, and any combination thereof;
(d) one or more genes selected from the group consisting of: ChyA, CRST-P, CTL1-LIKE, LGBP, PPAE2, CHYB, SP and any combination thereof;
(e) one or more genes selected from the group consisting of: CLT1-like, SP, CHYB, CRST-P, PPEA2, and CHYA;
(e) LGBP;
(f) one or more genes selected from the group consisting of: CRST-P, SP, PPEA2, CHYA, and LGBP;
(g) one or more genes selected from the group consisting of: SP, CRST-P, PPAE2, CHYA; or
(h) a combination thereof.

20. The modified non-human organism of claim 19, wherein the modified non-human organism is modified to comprise an AHPND tolerant signature.

21. The modified non-human organism of claim 20, wherein the AHPND tolerant signature comprises
(a) decreased expression of SP as compared to a suitable control and increased expression of CHYA, LGBP, and PPEA2 as compared to a suitable control;
(b) increased expression of CHYA, CRST-P, CTL1-like, LGBP, and PPAE2 as compared to a control sample obtained from an AHPND tolerant animal and decreased expression of CHYB and SP as compared to a control sample obtained from an AHPND tolerant animal;
(c) increased expression of PPAE2 and CHYA as compared to a suitable control and decreased expression of CTL1-like, CRST-P, SP, and CHYB as compared to a suitable control;
(d) increased expression of CRST-P, PPAE2, and CHYA as compared to a control sample obtained from an AHPND tolerant animal;
(e) decreased expression of LGBP as compared to a suitable control;
(f) increased expression of CHYB, and SP as compared to a control sample obtained from an AHPND susceptible animal and decreased expression of CTL1-like, CRST-P, PPAE2, LGBP and CHYA as compared to a control sample obtained from an AHPND susceptible animal;
(g) increased expression of PPEA2, CHYA, and LGBP as compared to a suitable control and decreased expression of CRST-P and SP as compared to a suitable control;
(h) increased expression of SP as compared to a control sample obtained from an AHPND susceptible animal and decreased expression of CRST-P, PPEA2, and CHYA as compared to a control sample obtained from an AHPND susceptible animal; or
(i) a combination thereof.

22. The modified non-human organism of claims 19-21, wherein the modified non-human organism is a crustacean.

23. The modified non-human organism of claim 22, wherein the crustacean is a crab, lobster, crayfish, shrimp, prawn, or krill.


SEQUENCE LISTING (34 sequences)

SEQ ID NO: 1 - protein, 15 aa, Artificial Sequence
  Synthetic glycine linker
GGGGGGGGGGGGGGG

SEQ ID NO: 2 - protein, 15 aa, Artificial Sequence
  Synthetic glycine serine linker
GGGGSGGGGSGGGGS

SEQ ID NO: 3 - protein, 5 aa, Artificial Sequence
  Synthetic pentapeptide ligand
TEFCA

SEQ ID NO: 4 - DNA, 23 bp, Artificial Sequence
  Synthetic primer
ccgtgcatatccgaattactcaa

SEQ ID NO: 5 - DNA, 20 bp, Artificial Sequence
  Synthetic primer
gcaccatcagggcgtttctc

SEQ ID NO: 6 - DNA, 21 bp, Artificial Sequence
  Synthetic primer
gctggcttacggctacgactt

SEQ ID NO: 7 - DNA, 24 bp, Artificial Sequence
  Synthetic primer
ggagaagaatgacgagtcggtcta

SEQ ID NO: 8 - DNA, 20 bp, Artificial Sequence
  Synthetic primer
agcgactgcaggtattggtg

SEQ ID NO: 9 - DNA, 19 bp, Artificial Sequence
  Synthetic primer
tcgttggaacaggttgtgg

SEQ ID NO: 10 - DNA, 20 bp, Artificial Sequence
  Synthetic primer
caagacccacagcggagaga

SEQ ID NO: 11 - DNA, 20 bp, Artificial Sequence
  Synthetic primer
tagacgagggcgaaggtttc

SEQ ID NO: 12 - DNA, 20 bp, Artificial Sequence
  Synthetic primer
aggcagctgtttgcacgaat

SEQ ID NO: 13 - DNA, 22 bp, Artificial Sequence
  Synthetic primer
agtgcagtcacaattgccatct

SEQ ID NO: 14 - DNA, 22 bp, Artificial Sequence
  Synthetic primer
catgtccaacttcgctttcaga

SEQ ID NO: 15 - DNA, 18 bp, Artificial Sequence
  Synthetic primer
atcaccgcgtggcatctt

SEQ ID NO: 16 - DNA, 21 bp, Artificial Sequence
  Synthetic primer
atgaagacgttggcaactctg

SEQ ID NO: 17 - DNA, 18 bp, Artificial Sequence
  Synthetic primer
ctcgcaggtggagtggag

SEQ ID NO: 18 - DNA, 22 bp, Artificial Sequence
  Synthetic primer
acgttctcacgactggtcacac

SEQ ID NO: 19 - DNA, 21 bp, Artificial Sequence
  Synthetic primer
tatgtaaggcgcgtcgttctc

SEQ ID NO: 20 - DNA, 19 bp, Artificial Sequence
  Synthetic primer
tcgtggtctgcctggtctt

SEQ ID NO: 21 - DNA, 21 bp, Artificial Sequence
  Synthetic primer
caggtctgaacggtggtcttc

SEQ ID NO: 22 - DNA, 19 bp, Artificial Sequence
  Synthetic primer
ttccttgggtggctgcttt

SEQ ID NO: 23 - DNA, 20 bp, Artificial Sequence
  Synthetic primer
tgttcgccgagacggattac

SEQ ID NO: 24 - DNA, 19 bp, Artificial Sequence
  Synthetic primer
agccagccaggtctccatt

SEQ ID NO: 25 - DNA, 24 bp, Artificial Sequence
  Synthetic primer
aagagttccagttctcgtgagtga

SEQ ID NO: 26 - DNA, 20 bp, Artificial Sequence
  Synthetic primer
accggcagtatctccaacgt

SEQ ID NO: 27 - DNA, 20 bp, Artificial Sequence
  Synthetic primer
ttgcagtcgtcgttcgtcat

SEQ ID NO: 28 - DNA, 19 bp, Artificial Sequence
  Synthetic primer
caaacggaggcgtcacaga

SEQ ID NO: 29 - DNA, 19 bp, Artificial Sequence
  Synthetic primer
gaccgacttccgggatgat

SEQ ID NO: 30 - DNA, 24 bp, Artificial Sequence
  Synthetic primer
tgcaaaccaagataacgtgtatga

SEQ ID NO: 31 - DNA, 19 bp, Artificial Sequence
  Synthetic primer
gccgtgaaccgtacaccaa

SEQ ID NO: 32 - DNA, 21 bp, Artificial Sequence
  Synthetic primer
tcgccgaactgctgaccaaga

SEQ ID NO: 33 - DNA, 20 bp, Artificial Sequence
  Synthetic primer
ccggcttccagttccttacc

SEQ ID NO: 34 - protein, 5 aa, Artificial Sequence
  Synthetic antimicrobial peptide
CRSTP